(19) United States
(12) Patent Application Publication
(10) Pub. No.: US 2017/0112941 A1
(43) Pub. Date: Apr. 27, 2017

(54) VE-CADHERIN BINDING BIOCONJUGATE

(71) Applicant: Symic IP, LLC, Emeryville, CA (US)

(72) Inventors: Alyssa Panitch, Davis, CA (US); John Eric Paderi, San Francisco, CA (US); Katherine Allison Stuart, San Francisco, CA (US); Seema Kantak, Pacifica, CA (US); Nathan Bachtell, San Francisco, CA (US)

(21) Appl. No.: 15/292,009

(22) Filed: Oct. 12, 2016

Related U.S. Application Data
(60) Provisional application No. 62/241,057, filed on Oct. 13, 2015.
(60) Provisional application No. 62/276,182, filed on Jan. 7, 2016.
(60) Provisional application No. 62/312,397, filed on Mar. 23, 2016.

(51) Int. Cl. A61K 38/00; A61K 31/728; A61K 31/727; A61K 31/737

(52) U.S. Cl. CPC A61K 47/48246; A61K 31/727; A61K 31/737; A61K 31/728; A61K 47/48338; A61K 47/48253

(57) ABSTRACT

This disclosure provides bioconjugate comprising a glycan and at least one peptide comprising a VE-Cadherin binding unit conjugated thereto, compositions comprising the same, and methods of use thereof.

### CROSS-REFERENCE TO RELATED APPLICATIONS

This application claims the benefit under 35 U.S.C. 119(e) to U.S. Provisional Application No. 62/241,057, filed Oct. 13, 2015, U.S. Provisional Application No. 62/276,182, filed Jan. 7, 2016, and U.S. Provisional Application No. 62/312,397, filed Mar. 23, 2016, where the contents of each is incorporated herein by reference in its entirety.

### BACKGROUND

Vascular endothelial (VE)-cadherin is an endothelial specific adhesion molecule located at junctions between endothelial cells. The endothelium of blood vessels provides a complex system of passively transporting tubes and also actively controls the entry of leukocytes and other substances into tissue. The control of endothelial cell-cell contacts is of vital importance for this process. VE-cadherin is a major determinant of the integrity of endothelial cell-cell contact and the regulation of its function at the junctions between endothelial cells is an essential step which controls the permeability of the blood vessel wall.

### SUMMARY

The present disclosure provides bioconjugates which bind to VE-cadherin, thus stabilizing endothelial cell-cell interactions.

In one embodiment, the present disclosure provides a bioconjugate comprising a glycan and at least one peptide comprising a VE-Cadherin binding unit. In certain embodiments, the peptide is derived from fibrin.

In one embodiment, provided herein is a bioconjugate comprising a glycan and at least one peptide comprising an amino acid sequence PSLRPAPPPISGGGYR (SEQ ID NO:1), or an amino acid sequence having one, two, or three amino acid addition, deletion and/or substitution(s) therefrom. In one embodiment, provided herein is a bioconjugate comprising a glycan and at least one peptide comprising an amino acid sequence GHRPLDKKREEAPSLRPA (SEQ ID NO:2), or an amino acid sequence having one, two, or three amino acid addition, deletion and/or substitution(s) therefrom. In another embodiment, provided herein is a bioconjugate comprising a glycan and at least one peptide comprising an amino acid sequence GHRPLDKKREEAPSLRPAPPPISGGGYR (SEQ ID NO:3), or an amino acid sequence having one, two, or three amino acid addition, deletion and/or substitution therefrom.

In one embodiment, the bioconjugate further comprises at least one selectin-binding unit, ICAM-binding unit, VCAM-binding unit, and/or collagen-binding unit.

The number of available binding sites for peptide conjugation can also vary depending on the structure of the glycan which is employed, and thus the number of peptides bound to the glycan can vary. The glycan can be any glycan, such as, but not limited to, alginate, chondroitin, chondroitin sulfate, dermatan, dermatan sulfate, heparan, heparan sulfate, heparin, dextran, dextran sulfate, and hyaluronan, or a derivative thereof. In certain embodiments, the bioconjugate comprises from about 1 to about 100 peptides, or from about 5 to about 80 peptides, or from about 50 to about 80 peptides, or from about 60 to about 70 peptides, or from 1 to about 25 peptides, or from about 5 to about 25 peptides, or from about 1 to about 15 peptides, or about 2 peptides, or about 5 peptides, or about 10 peptides, or about 15 peptides, or about 20 peptides, or about 30 peptides, or about 40 peptides, or about 50 peptides, or about 60 peptides, or about 70 peptides, or about 80 peptides per glycan. In certain embodiments, the glycan comprises: a) from about 1 to about 75 percent (%) functionalization, b) from about 5 to about 30 percent (%) functionalization, c) from about 10 to about 40 percent (%) functionalization, d) about 25 percent (%) functionalization, or e) about 30 percent (%) functionalization, wherein the percent (%) functionalization is determined by a percent of disaccharide units on the glycan which are functionalized with peptide. In certain embodiments, the peptide is bound to the glycan via a spacer. In some embodiments, the spacer comprises between about 5 to about 50 carbon atoms. In some embodiments, the spacer is branched.

Also provided herein is a composition comprising a bioconjugate as described herein and one or more bioconjugates selected from the group consisting of a) a bioconjugate comprising a glycan and at least one peptide comprising a selectin-binding unit; b) a bioconjugate comprising a glycan and at least one peptide comprising a ICAM-binding unit; c) a bioconjugate comprising a glycan and at least one peptide comprising a VCAM-binding unit; and d) a bioconjugate comprising a glycan and at least one peptide comprising a collagen-binding unit. In certain embodiments, provided is a composition comprising a bioconjugate as described herein and a bioconjugate comprising a glycan and at least one peptide comprising a collagen-binding unit.

Also provided herein is a composition comprising a bioconjugate as described herein wherein the average number of peptide(s) per glycan is about 80, or about 70, or about 60, or about 50, or about 40, or about 30, or less than about 30, or about 5 to about 25, or about 5, or about 8, or about 10. In certain embodiments, provided is a composition comprising a bioconjugate as described herein wherein the average number of peptide(s) per glycan is about 70.

Also provided herein are pharmaceutical compositions comprising a bioconjugate as described herein, or a composition comprising the same, and one or more pharmaceutically acceptable diluents or carriers.

Also provided herein is a method for maintaining endothelial integrity in a patient in need thereof, comprising administering to the patient an effective amount of a bioconjugate as described herein, or a composition comprising the same.

Also provided herein is a method for treating a patient suffering from a disease associated with endothelial dysfunction, the method comprising administering to the patient an effective amount of a bioconjugate as described herein, or a composition comprising the same. Non-limiting examples of diseases associated with endothelial dysfunction is selected from the group consisting of atherosclerosis, coronary artery disease, myocardial infarction, diabetes mellitus, hypertension, hypercholesterolemia, rheumatoid arthritis, systemic lupus erythematosus, glaucoma, uremia, sepsis, organ failure, shock, Dengue viral infection, acute lung injury, and acute kidney injury. In certain embodiments, the treating comprises cardiac reperfusion following myocardial infarction.

Also provided herein is a method for preventing or reducing inflammation at a vascular site in a patient, the method comprising administering to the patient an effective amount of a bioconjugate as described herein, or a composition comprising the same. In certain embodiments, the site (a) comprises permeated endothelial lining or damaged endothelial cells, and (b) is not undergoing or recovering from a vascular intervention procedure. In one embodiment, the vascular intervention procedure comprises a percutaneous coronary intervention (PCI) procedure.

The present disclosure further provides methods for treating or preventing ischemic reperfusion injury in a patient in need thereof, comprising administering to the patient an effective amount of a bioconjugate as described herein, or a composition comprising the same. In one embodiment, the ischemic reperfusion injury is a result of organ transplant, such as the kidney, heart, liver, or a vein graft. The organ can be perfused with the bioconjugate or composition at any time, including, but not limited to, just prior to, at the time of, and/or periodically after reperfusion.

The present disclosure further provides methods for inhibiting and/or treating fibrosis by administering a bioconjugate as described herein, or a composition comprising the same. In certain embodiments, the fibrosis is the result of a fibrotic disease, such as, but not limited to, pulmonary fibrosis, cystic fibrosis, idiopathic pulmonary fibrosis, renal fibrosis, cirrhosis, cardiac fibrosis, atrial fibrosis, endomyocardial fibrosis, myocardial infarction, glial scar, arthrofibrosis, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma, systemic sclerosis and/or adhesive capsulitis.

Also provided are methods for inhibiting and/or treating fibrosis by administering a bioconjugate as described herein in combination another anti-fibrotic agent. Non-limiting examples include predonine, N-acetylcysteine, pirfenidone, nintedanib, corticosteroids, cyclophosphamide, azathioprine, methotrexate, penicillamine, cyclosporine A, FK506, colchicine, IFN-γ and mycophenolate mofetil.

### BRIEF DESCRIPTION OF THE DRAWINGS

FIG. 1 is a schematic showing the role of VE-cadherin in treating fibrosis by mitigating inflammation caused by endothelial cell-cell barrier loss, and subsequent leukocyte extravasation.

FIG. 2 shows that the bioconjugate described herein binds to VE-cadherin in a dose dependent manner.

FIG. 3 shows that the bioconjugate described herein preserves endothelial cell barrier function.

FIG. 4 shows that the bioconjugate as described in Example 1 protects from renal damage upon reperfusion better than active control (peptide alone) in an acute renal ischemic model.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### 1. DEFINITIONS

Unless defined otherwise, all technical and scientific terms used herein have the same meaning as commonly understood by one of ordinary skill in the art to which this disclosure belongs. As used herein the following terms have the following meanings.

It must be noted that as used herein and in the appended claims, the singular forms “a”, “an”, and “the” include plural referents unless the context clearly dictates otherwise. Thus, for example, reference to “a peptide” includes a plurality of peptides.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The following abbreviations used herein have the following meanings.

| | |
|---|---|
| ° C. | Degrees Celsius |
| μg | Microgram |
| BMPH | N-[β-maleimidopropionic acid]hydrazide |
| BMPH-CS | BMPH Linker-Chondroitin Sulfate Conjugate |
| cps | Centipoise |
| CS | Chondroitin sulfate |
| Dex | Dextran |
| DNA | Deoxyribonucleic acid |
| DS | Dermatan Sulfate |
| ECM | Extracellular Matrix |
| EDTA | Ethylenediaminetetraacetic Acid |
| ELISA | Enzyme-Linked Immunosorbent Assay |
| GAG | Glycosaminoglycan |
| Hep | Heparin |
| HLB | Hydrophile/Lipophile/Balance |
| HPC | Hydroxyl Propylcellulose |
| ITC | Isothermal Titration Calorimeters |
| kDa | KiloDalton |
| kg | Kilogram |
| MES | 2-ethanesulfonic acid |
| mg | Milligram |
| mL | Milliliter |
| mOsm | Milliosmole |
| mV | Millivolt |
| ng | Nanogram |
| PBS | Phosphate buffered saline |
| QD | Administered Once Daily |
| SPR | Surface Plasmon Resonance |
| TAPS | 3-[[1,3-dihydroxy-2-(hydroxymethyl)propan-2- |
| | yl]amino]propane-1-sulfonic acid |
| TES | 2-[[1,3-dihydroxy-2-(hydroxymethyl)propan-2- |
| | yl]amino]ethanesulfonic acid |
| Tris | 2-Amino-2-hydroxymethyl-propane-1,3-diol |
| w/w | Weight/Weight |
| w/v | Weight/Volume |

As used herein, the term “treating” refers to preventing, curing, reversing, attenuating, alleviating, minimizing, inhibiting, suppressing and/or halting one or more clinical symptoms of a disease or disorder in a patient suffering therefrom prior to, during, and/or after a medical intervention (such as organ transplant).

As used herein, the term “pharmaceutical composition” refers to a preparation suitable for administration to an intended patient for therapeutic purposes that contains at least one pharmaceutically active ingredient, including any solid form thereof. The pharmaceutical composition may include at least one pharmaceutically acceptable component to provide an improved formulation of the compound, such as a suitable carrier. In certain embodiments, the pharmaceutical composition is formulated as a film, gel, patch, or liquid solution. As used herein, the term “topically” refers to administering a pharmaceutical composition non-systemically to the surface of a tissue and/or organ (internal or, in some cases, external; through a catheter) to be treated, for local effect.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue.

As used herein, the term “formulated” or “formulation” refers to the process in which different chemical substances, including one or more pharmaceutically active ingredients, are combined to produce a dosage form. In certain embodiments, two or more pharmaceutically active ingredients can be coformulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “delivery” refers to approaches, formulations, technologies, and systems for transporting a pharmaceutical composition in the body as needed to safely achieve its desired therapeutic effect. In some embodiments, an effective amount of the pharmaceutical composition is formulated for delivery into the blood stream of a patient.

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

As used herein, the term “polymer matrix” or “polymeric agent” refers to a biocompatible polymeric materials. The polymeric material described herein may comprise, for example, sugars (such as mannitol), peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

As used herein, the term “absorbable” refers to the ability of a material to be absorbed into the body. In certain embodiments, the polymeric matrix is absorbable, such as, for example collagen, polyglycolic acid, polylactic acid, polydioxanone, and caprolactone. In certain embodiments, the polymer is non-absorbable, such as, for example polypropylene, polyester or nylon.

As used herein, the term “pH buffering agent” refers to an aqueous buffer solution which resists changes in pH when small quantities of acid or base are added to it. pH Buffering solutions typically comprise of a mixture of weak acid and its conjugate base, or vice versa. For example, pH buffering solutions may comprise phosphates such as sodium phosphate, sodium dihydrogen phosphate, sodium dihydrogen phosphate dihydrate, disodium hydrogen phosphate, disodium hydrogen phosphate dodecahydrate, potassium phosphate, potassium dihydrogen phosphate and dipotassium hydrogen phosphate; boric acid and borates such as, sodium borate and potassium borate; citric acid and citrates such as sodium citrate and disodium citrate; 7 acetates such as sodium acetate and potassium acetate; carbonates such as sodium carbonate and sodium hydrogen carbonate, etc. pH Adjusting agents can include, for example, acids such as hydrochloric acid, lactic acid, citric acid, phosphoric acid and acetic acid, and alkaline bases such as sodium hydroxide, potassium hydroxide, sodium carbonate and sodium hydrogen carbonate, etc. In some embodiments, the pH buffering agent is a phosphate buffered saline (PBS) solution (i.e., containing sodium phosphate, sodium chloride and in some formulations, potassium chloride and potassium phosphate).

### 2. BIOCONJUGATES

As used herein, the term “bioconjugate” refers to a conjugate that comprises a glycan and one or more synthetic peptides conjugated, via a covalent bond, thereto. The glycan portion can be made synthetically or derived from animal sources. The synthetic peptides can be covalently bonded directly to the glycan or via a linker. For methods of conjugating the peptides as described herein to a glycan, see, e.g., US 2013/0190246, US 2012/0100106, and US 2011/0020298, the disclosures of which are incorporated herein by reference in their entirety. In one embodiment, the molecular weight range for the bioconjugate is from about 13 kDA to about 1.2 MDa, or from about 500 kDa to about 1 MDa, or from about 20 kDa to about 90 kDa, or from about 10 kDa to about 70 kDa.

In one embodiment, the bioconjugates of the disclosure bind, either directly or indirectly to hyaluronic acid (HA), collagen, ECM, or endothelium. The terms “binding” or “bind” as used herein are meant to include interactions between molecules that may be detected using, for example, a hybridization assay, surface plasmon resonance, ELISA, competitive binding assays, isothermal titration calorimetry, phage display, affinity chromatography, rheology or immunohistochemistry. The terms are also meant to include “binding” interactions between molecules. Binding may be “direct” or “indirect.” “Direct” binding comprises direct physical contact between molecules. “Indirect” binding between molecules comprises the molecules having direct physical contact with one or more molecules simultaneously. This binding can result in the formation of a “complex” comprising the interacting molecules. A “complex” refers to the binding of two or more molecules held together by covalent or non-covalent bonds, interactions or forces.

Peptides

The peptides of the bioconjugates can be synthesized and evaluated for binding to the target (e.g., VE-cadherin) by any of the techniques such as SPR, ELISA, ITC, affinity chromatography, or others known in the art. An example could be a biotin modified peptide sequence that is incubated on a microplate containing immobilized VE-cadherin. A dose response binding curve can be generated using a streptavidin-chromophore to determine the ability of the peptide to bind to VE-cadherin. In various embodiments described herein, the peptides described herein can be modified by the inclusion of one or more conservative amino acid substitutions. As is well known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced. Non-conservative substitutions may too be possible, provided that they do not substantially affect the binding activity of the sequence (i.e., VE-cadherin-binding affinity).

As used herein, the term “sequence identity” refers to a level of amino acid residue or nucleotide identity between two peptides or between two nucleic acid molecules. When a position in the compared sequence is occupied by the same base or amino acid, then the molecules are identical at that position. A peptide (or a polypeptide or peptide region) has a certain percentage (for example, at least about 60%, or at least about 65%, or at least about 70%, or at least about 75%, or at least about 80%, or at least about 83%, or at least about 85%, or at least about 90%, or at least about 95%, or at least about 98% or at least about 99%) of “sequence identity” to another sequence means that, when aligned, that percentage of bases (or amino acids) are the same in comparing the two sequences. It is noted that, for any sequence (“reference sequence”) disclosed in this application, sequences having at least about 60%, or at least about 65%, or at least about 70%, or at least about 75%, or at least about 80%, or at least about 83%, or at least about 85%, or at least about 90%, or at least about 95%, or at least about 98% or at least about 99% sequence identity to the reference sequence are also within the disclosure. Likewise, the present disclosure also includes sequences that have one, two, three, four, or five substitution, deletion or addition of amino acid residues or nucleotides as compared to the reference sequences.

As is well-known in the art, a “conservative substitution” of an amino acid or a “conservative substitution variant” of a peptide refers to an amino acid substitution which maintains: 1) the secondary structure of the peptide; 2) the charge or hydrophobicity of the amino acid; and 3) the bulkiness of the side chain or any one or more of these characteristics. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, ornithine, or histidine. “Negatively charged residues” refer to aspartic acid or glutamic acid. Residues having “bulky side chains” refer to phenylalanine, tryptophan or tyrosine, or the like. A list of illustrative conservative amino acid substitutions is given in Table 1.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L- |
| | Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

### VE-Cadherin Binding Peptides

“VE-cadherin binding peptides” are peptides, typically from 1 to about 120 amino acids, comprising one or more VE-cadherin binding units (or sequences). As used herein, the term “VE-Cadherin binding unit” is intended to refer to an amino acid sequence which binds to the endothelial cell adhesion molecule, VE-cadherin. “VE-cadherin binding” indicates an interaction with VE-cadherin that could include hydrophobic, ionic (charge), and/or Van der Waals interactions, such that the compound binds or interacts favorably with VE-cadherin. This binding (or interaction) is intended to be differentiated from covalent bonds and nonspecific interactions with common functional groups, such that the peptide would interact with any species containing that functional group to which the peptide binds on the VE-cadherin. Peptides can be tested and assessed for binding to VE-cadherin using any method known in the art. See, e.g., Gorlatov, S., Biochemistry, 2002, 41(12), 4107-4116, Yakovlev, S., J Thromb Haemost, 2011, 9, 1847-55 and Heupel, W. M., J Cell Sci., 2009, 122(Pt 10), 1616-25. In one embodiment, the peptide, or the VE-cadherin binding unit of the peptide, binds to VE-cadherin with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM.

In certain embodiments, the peptide is fibrin or a fibrin derivative which comprises one or more VE-cadherin binding units.

In certain embodiments, the VE-cadherin binding unit comprises one or more sequences selected from the group consisting of PSLRPAPPPISGGGYR (SEQ ID NO:1), APSLRPAPPPISGGGYR (SEQ ID NO:5), AAPSLRPAPPPISGGGYR (SEQ ID NO:6), RAAPSLRPAPPPISGGGYR (SEQ ID NO:7), PSLRPAPPPISGGGYRGSG (SEQ ID NO:8), APSLRPAPPPISGGGYRGSG (SEQ ID NO:9), AAPSLRPAPPPISGGGYRGSG (SEQ ID NO:10), and RAAPSLRPAPPPISGGGYRGSG (SEQ ID NO:11), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin.

In certain embodiments, the VE-cadherin binding unit comprises one or more cyclic peptide sequences selected from the group consisting of CRVDAE-Ahx-RVDAEC (SEQ ID NO:12) or CRVDAE-Ahx-RVDAECGSG (SEQ ID NO:13), wherein the peptide is cyclized at the cysteines and Ahx is 6-aminohexanoic acid, or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin. In certain embodiments, the VE-cadherin binding unit comprises one or more cyclic peptide sequences selected from the group consisting of CRVDAE-Ahx-RVDAEC (SEQ ID NO:12) or CRVDAE-Ahx-RVDAECGSG (SEQ ID NO:13), wherein the peptide is cyclized at the cysteines and Ahx is 6-aminohexanoic acid, or an amino acid sequence having one, two, or three amino acid addition, deletion and/or substitution therefrom.

In certain embodiments, the VE-cadherin binding unit comprises GHRPLDKKREEAPSLRPAPPPISGGGYR (SEQ ID NO:3), GHRPLDKKREEAPSLRPAPPPISGGGYRGSG (SEQ ID NO:14), or a sequence having at least about 70% sequence identity, or at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin.

In certain embodiments, the VE-cadherin binding unit comprises GHRPLDKKREEAPSLRPAPPPISGGGYR (SEQ ID NO:3). Accordingly, provided herein is a bioconjugate comprising a glycan and at least one peptide comprising GHRPLDKKREEAPSLRPAPPPISGGGYR (SEQ ID NO:3). In one embodiment, provided herein is a bioconjugate comprising heparin and from 1 to about 10 peptides comprising GHRPLDKKREEAPSLRPAPPPISGGGYR (SEQ ID NO:3). In one embodiment, provided herein is a bioconjugate comprising heparin and about 5 peptides comprising GHRPLDKKREEAPSLRPAPPPISGGGYR (SEQ ID NO:3). In another embodiment, provided herein is a bioconjugate comprising dermatan sulfate and from 1 to about 15 peptides comprising GHRPLDKKREEAPSLRPAPPPISGGGYR (SEQ ID NO:3). In certain embodiments, the peptides are bound to the glycan (e.g., heparin, dermatan sulfate, etc.) via a hydrazide-carbonyl bond.

In certain embodiments, the VE-cadherin binding unit comprises the sequence GHRPLDKKREEAPSLRPAPPPISGGGYR (SEQ ID NO:3) or GHRPLDKKREEAPSLRPAPPPISGGGYRGSG (SEQ ID NO:14), or a truncated version thereof, wherein one or more amino acids has been deleted, or from 1 to 10, or from 1 to 9, or from 1 to 8, or from 1 to 7, or from 1 to 6, or from 1 to 5, or from 1 to 4, or from 1 to 3, or 1 to 2 amino acids have been deleted, provided the sequence is capable of binding to VE-cadherin. For example, in certain embodiments, the binding unit comprises a sequence selected from the group consisting of GHRPLDKKREEAPSLRPAPPPISGGGY (SEQ ID NO:15), GHRPLDKKREEAPSLRPAPPPISGGG (SEQ ID NO:16), GHRPLDKKREEAPSLRPAPPPISGG (SEQ ID NO:17), GHRPLDKKREEAPSLRPAPPPISG (SEQ ID NO:18), GHRPLDKKREEAPSLRPAPPPIS (SEQ ID NO:19), GHRPLDKKREEAPSLRPAPPPI (SEQ ID NO:20), GHRPLDKKREEAPSLRPAPPP (SEQ ID NO:21), GHRPLDKKREEAPSLRPAPP (SEQ ID NO:22), GHRPLDKKREEAPSLRPAP (SEQ ID NO:23), GHRPLDKKREEAPSLRPA (SEQ ID NO:2), GHRPLDKKREEAPSLRP (SEQ ID NO:24), GHRPLDKKREEAPSLR (SEQ ID NO:25), GHRPLDKKREEAPSL (SEQ ID NO:26), GHRPLDKKREEAPS (SEQ ID NO:27), GHRPLDKKREEAP (SEQ ID NO:28), GHRPLDKKREEA (SEQ ID NO:29), GHRPLDKKREE (SEQ ID NO:30), GHRPLDKKRE (SEQ ID NO:31), and GHRPLDKKR (SEQ ID NO:32), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin.

In certain embodiments, the VE-cadherin binding unit comprises GHRPLDKKREEAPSLRPA (SEQ ID NO:2) or GHRPLDKKREEAPSLRPAGSG (SEQ ID NO:33), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin. In certain embodiments, the VE-cadherin binding unit comprises GHRPLDKKREEAPSLRPA (SEQ ID NO:2) or GHRPLDKKREEAPSLRPAGSG (SEQ ID NO:33), or an amino acid sequence having one, two, or three amino acid addition, deletion and/or substitution therefrom.

In certain embodiments, any sequence described herein may be modified such that any one or more amino acids (e.g., 1, 2, 3, 4 or 5 amino acids) are added, deleted or substituted therefrom. In some embodiments, the sequence is modified such that any one or more amino acids is replaced by alanine. In some embodiments, the sequence is modified such that any one or more 1-amino acid is replaced the corresponding d-amino acid scan. In some embodiments, the sequence is modified such that any one or more valine is replaced by leucine, any one or more glutamic acid is replaced by glutamine, any one or more aspartic acid is replaced by asparagine, and/or any one or more arginine is replaced by glutamine.

Accordingly, in certain embodiments, the VE-cadherin binding unit is a sequence selected from the group consisting of XHRPLDKKREEAPSLRPA (SEQ ID NO:34), GXRPLDKKREEAPSLRPA (SEQ ID NO:35), GHXPLDKKREEAPSLRPA (SEQ ID NO:36), GHRXLDKKREEAPSLRPA (SEQ ID NO:37), GHRPXDKKREEAPSLRPA (SEQ ID NO:38), GHRPLXKKREEAPSLRPA (SEQ ID NO:39), GHRPLDXKREEAPSLRPA (SEQ ID NO:40), GHRPLDKXREEAPSLRPA (SEQ ID NO:41), GHRPLDKKXEEAPSLRPA (SEQ ID NO:42), GHRPLDKKRXEAPSLRPA (SEQ ID NO:43), GHRPLDKKREXAPSLRPA (SEQ ID NO:44), GHRPLDKKREEXASLRPA (SEQ ID NO:45), GHRPLDKKREEAXSLRPA (SEQ ID NO:46), GHRPLDKKREEAPXLRPA (SEQ ID NO:47), GHRPLDKKREEAPSXRPA (SEQ ID NO:48), GHRPLDKKREEAPSLXPA (SEQ ID NO:49), GHRPLDKKREEAPSLRXA (SEQ ID NO:50), and GHRPLDKKREEAPSLRAX (SEQ ID NO:51), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, wherein X is absent or a natural or unnatural amino acid and wherein the sequence is capable of binding to VE-cadherin.

In certain embodiments, X is arginine. In certain embodiments, X is alanine, and the VE-cadherin binding unit is a sequence selected from the group consisting of AHRPLDKKREEAPSLRPA (SEQ ID NO:52), GARPLDKKREEAPSLRPA (SEQ ID NO:53), GHAPLDKKREEAPSLRPA (SEQ ID NO:54), GHRALDKKREEAPSLRPA (SEQ ID NO:55), GHRPADKKREEAPSLRPA (SEQ ID NO:56), GHRPLAKKREEAPSLRPA (SEQ ID NO:57), GHRPLDAKREEAPSLRPA (SEQ ID NO:58), GHRPLDKAREEAPSLRPA (SEQ ID NO:59), GHRPLDKKAEEAPSLRPA (SEQ ID NO:60), GHRPLDKKRAEAPSLRPA (SEQ ID NO:61), GHRPLDKKREAAPSLRPA (SEQ ID NO:62), GHRPLDKKREEAASLRPA (SEQ ID NO:63), GHRPLDKKREEAPALRPA (SEQ ID NO:64), GHRPLDKKREEAPSARPA (SEQ ID NO:65), GHRPLDKKREEAPSLAPA (SEQ ID NO:66), and GHRPLDKKREEAPSLRAA (SEQ ID NO:67), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin.

In certain embodiments, any one or more glutamic acid is replaced by glutamine, any one or more aspartic acid is replaced by asparagine, and/or any one or more arginine is replaced by glutamine. Accordingly, in certain embodiments, the VE-cadherin binding unit is a sequence selected from the group consisting of GHRPLNKKREEAPSLRPA (SEQ ID NO:68), GHRPLDKKRQEAPSLRPA (SEQ ID NO:69), GHRPLDKKREQAPSLRPA (SEQ ID NO:70), GHRPLDKKRQQAPSLRPA (SEQ ID NO:71), GHRPLNKKRQEAPSLRPA (SEQ ID NO:72), GHRPLNKKREQAPSLRPA (SEQ ID NO:73), and GHRPLNKKRQQAPSLRPA (SEQ ID NO:74), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin.

In certain embodiments, the VE-cadherin binding unit may be modified such that any one or more 1-amino acid is replaced by the corresponding d-amino acid. Accordingly, in certain embodiments, the VE-cadherin binding unit is a sequence selected from the group consisting of gHRPLDKKREEAPSLRPA (SEQ ID NO:2), GHRPLDKKREEAPSLRPA (SEQ ID NO:2), GHrPLDKKREEAPSLRPA (SEQ ID NO:2), GHRpLDKKREEAPSLRPA (SEQ ID NO:2), GHRPlDKKREEAPSLRPA (SEQ ID NO:2), GHRPLdKKREEAPSLRPA (SEQ ID NO:2), GHRPLDkKREEAPSLRPA (SEQ ID NO:2), GHRPLDKkREEAPSLRPA (SEQ ID NO:2), GHRPLDKKrEEAPSLRPA (SEQ ID NO:2), GHRPLDKKReEAPSLRPA (SEQ ID NO:2), GHRPLDKKREeAPSLRPA (SEQ ID NO:2), GHRPLDKKREEaPSLRPA (SEQ ID NO:2), GHRPLDKKREEApSLRPA (SEQ ID NO:2), GHRPLDKKREEAPsLRPA (SEQ ID NO:2), GHRPLDKKREEAPSlRPA (SEQ ID NO:2), GHRPLDKKREEAPSLrPA (SEQ ID NO:2), GHRPLDKKREEAPSLRpA (SEQ ID NO:2), GHRPLDKKREEAPSLRPa (SEQ ID NO:2), GHRPLDKKREEAPSLRPA (SEQ ID NO:2), GHRPLDKKREEAPSLRPA (SEQ ID NO:2), GHRPLDkkREEAPSLRPA (SEQ ID NO:2), GHRPLDkkrEEAPSLRPA (SEQ ID NO:2), GHRPLDkkREEAPSLrPA (SEQ ID NO:2), GHrPLDKKREEAPSLRPA (SEQ ID NO:2), GHrPLDKKrEEAPSLrPA (SEQ ID NO:2), and GHrPLDkkrEEAPSLrPA (SEQ ID NO:2), or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin, wherein the lowercase letter indicates an amino acid which has been replaced by the corresponding d-amino acid.

In addition, a VE-cadherin binding peptide derived from a phage display library selected for VE-cadherin can be generated. The peptide can be synthesized and evaluated for binding to VE-cadherin by any of the techniques such as SPR, ELISA, ITC, affinity chromatography, or others known in the art. An example could be a biotin modified peptide sequence that is incubated on a microplate containing immobilized VE-cadherin. A dose response binding curve can be generated using a streptavidin-chromophore to determine the ability of the peptide to bind to VE-cadherin.

### Collagen-Binding Peptides

“Collagen-binding peptides” are peptides comprising 1 to about 120 amino acids having one or more collagen-binding units (or sequences). As used herein, the term “collagen-binding unit” is intended to refer to an amino acid sequence within a peptide which binds to collagen. “Collagen-binding” indicates an interaction with collagen that could include hydrophobic, ionic (charge), and/or Van der Waals interactions, such that the compound binds or interacts favorably with collagen. This binding (or interaction) is intended to be differentiated from covalent bonds and nonspecific interactions with common functional groups, such that the peptide would interact with any species containing that functional group to which the peptide binds on the collagen. Peptides can be tested and assessed for binding to collagen using any method known in the art. See, e.g., Li, Y., et al., Current Opinion in Chemical Biology, 2013, 17: 968-975, Helmes, B. A., et al., J. Am. Chem. Soc. 2009, 131, 11683-11685, and Petsalaki, E., et al., PLoS Comput Biol, 2009, 5(3): e1000335. In one embodiment, the peptide, or the collagen-binding unit of the peptide, binds to collagen with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM.

Collagen-binding peptides can bind to one or more of collagen type I, II, III, IV, V, VI, VII, VIII, IX, X, XI, XII, XIII, or XIV. In some embodiments, the collagen-binding peptides bind to type IV collagen, which can be intact, cleaved or degraded. In some embodiments, the collagen-binding peptides bind to type I or III collagen, which can be intact, cleaved or degraded.

A non-limiting example of collagen-binding units that bind type IV collagen is TLTYTWS (SEQ ID NO:75) which binds specifically to MMP 2 and 9-degraded basement membrane type IV collagen. Likewise, TLTYTWSGSG (SEQ ID NO:76) which further includes a GSG linker can also bind to cleaved or degraded type IV collagen specifically. Another example is KLWVLPK (SEQ ID NO:77) which selectively binds to intact type IV collagen.

In various embodiments, the peptides that bind to type I or II collagen include an amino acid sequence selected from RRANAALKAGELYKSILY (SEQ ID NO:78), GELYKSILY (SEQ ID NO:79), RRANAALKAGELYKCILY (SEQ ID NO:80), GELYKCILY (SEQ ID NO:81), RLDGNEIKR (SEQ ID NO:82), AHEEISTTNEGVM (SEQ ID NO:83), NGVFKYRPRYFLYKHAYFYPPLKRFPVQ (SEQ ID NO:84), CQDSETRTFY (SEQ ID NO:85), TKKTLRT (SEQ ID NO:86), GLRSKSKKFRRPDIQYPDATDEDITSHM (SEQ ID NO:87), SQNPVQP (SEQ ID NO:88), SYIRIADTNIT (SEQ ID NO:89), KELNLVYT (SEQ ID NO:90), GSIT (SEQ ID NO:91), GSITTIDVPWNV (SEQ ID NO:92), GQLYKSILY (SEQ ID NO:93), RRANAALKAGQLYKSILY (SEQ ID NO:94), or a sequence having at least about 80% sequence identity, or at least about 83% sequence identity, or at least about 85% sequence identity, or at least about 90% sequence identity, or at least about 95% sequence identity, or at least about 98% sequence identity thereto, provided the sequence is capable of binding to collagen.

In certain embodiments, the peptide comprises an amino acid sequence that has at least about 80%, or at least about 83%, or at least about 85%, or at least about 90%, or at least about 95%, or at least about 98%, or at least about 100% sequence identity with the collagen-binding domain(s) of the von Willebrand factor (vWF) or a platelet collagen receptor as described in Chiang, T. M., et al. J. Biol. Chem., 2002, 277:00:00 34896-34901, Huizinga, E. G. et al., Structure, 1997, 5:00 1147-1156, Romijn, R. A., et al., J. Biol. Chem., 2003, 278:00:00 15035-15039, and Chiang, et al., Cardio. & Haemato. Disorders-Drug Targets, 2007, 7:00 71-75, each incorporated herein by reference. A non-limiting example is WREPSFCALS (SEQ ID NO:95), derived from vWF.

Various methods for screening peptide sequences for collagen-binding affinity (or a collagen-binding domain/unit) are routine in the art. Other peptide sequences shown to have collagen-binding affinity (or a collagen-binding unit) which can be used in the bioconjugates and methods disclosed herein include but are not limited to, βAWHCTTKFPHHYCLYBip (SEQ ID NO:96), βAHKCPWHLYTTHYCFTBip (SEQ ID NO:97), βAHKCPWHLYTHYCFT (SEQ ID NO:98), etc., where Bip is biphenylalanine and βA is beta-alanine (see, Abd-Elgaliel, W. R., et al., Biopolymers, 2013, 100(2), 167-173), GROGER (SEQ ID NO:99), GMOGER (SEQ ID NO:100), GLOGEN (SEQ ID NO:101), GLOGER (SEQ ID NO:102), GLKGEN (SEQ ID NO:103), GFOGERGVEGPOGPA (SEQ ID NO:104), etc., where O is 4-hydroxyproline (see, Raynal, N., et al., J. Biol. Chem., 2006, 281(7), 3821-3831), HVWMQAPGGGK (SEQ ID NO:105) (see, Helms, B. A., et al., J. Am. Chem. Soc. 2009, 131, 11683-11685), WREPSFCALS (SEQ ID NO:95) (see, Takagi, J., et al., Biochemistry, 1992, 31, 8530-8534), WYRGRL (SEQ ID NO:106), etc. (see, Rothenfluh D. A., et al., Nat Mater. 2008, 7(3), 248-54), WTCSGDEYTWHC (SEQ ID NO:107), WTCVGDHKTWKC (SEQ ID NO:108), QWHCTTRFPHHYCLYG (SEQ ID NO:109), etc. (see, U.S. 2007/0293656), STWTWNGSAWTWNEGGK (SEQ ID NO:110), STWTWNGTNWTRNDGGK (SEQ ID NO:111), etc. (see, WO/2014/059530), CVWLWEQC (SEQ ID NO:112) cyclic CVWLWENC (SEQ ID NO:113), cyclic CVWLWEQC (SEQ ID NO:112), (see, Depraetere H., et al., Blood. 1998, 92, 4207-4211, and Duncan R., Nat Rev Drug Discov, 2003, 2(5), 347-360), CMTSPWRC (SEQ ID NO:114), etc. (see, Vanhoorelbeke, K., et al., J. Biol. Chem., 2003, 278, 37815-37821), CPGRVMHGLHLGDDEGPC (SEQ ID NO:115) (see, Muzzard, J., et al., PLoS one. 4 (e 5585) I-10), KLWLLPK (SEQ ID NO:116) (see, Chan, J. M., et al., Proc Natl Acad Sci U.S.A., 2010, 107, 2213-2218), and CQDSETRTFY (SEQ ID NO:85), etc. (see, U.S. 2013/0243700), H—V—F/W-Q/M-Q-P/A-P/K (Helms, B. A., et al., J. Am. Chem. Soc., 2009, 131(33), 11683-11685), wherein each is hereby incorporated by reference in its entirety.

Additional peptide sequences shown to have collagen-binding affinity (or a collagen-binding unit) which can be used in the bioconjugates and methods disclosed herein include but are not limited to, LSELRLHEN (SEQ ID NO:117), LTELHLDNN (SEQ ID NO:118), LSELRLHNN (SEQ ID NO:119), LSELRLHAN (SEQ ID NO:120), and LRELHLNNN (SEQ ID NO:121) (see, Fredrico, S., Angew. Chem. Int. Ed. 2015, 37, 10980-10984).

In certain embodiments, the peptides include one or more sequences selected from the group consisting of RVMHGLHLGDDE (SEQ ID NO:122), D-amino acid EDDGLHLGHMVR (SEQ ID NO:123), RVMHGLHLGNNQ (SEQ ID NO:124), D-amino acid QNNGLHLGHMVR (SEQ ID NO:125), RVMHGLHLGNNQ (SEQ ID NO:124), (GQLYKSILYGSG)₄K₂K (SEQ ID NO:126) (a 4-branch peptide), GSGQLYKSILY (SEQ ID NO:127), GSGGQLYKSILY (SEQ ID NO:128), KQLNLVYT (SEQ ID NO:129), CVWLWQQC (SEQ ID NO:130), WREPSFSALS (SEQ ID NO:131), GHRPLDKKREEAPSLRPAPPPISGGGYR (SEQ ID NO:3), and GHRPLNKKRQQAPSLRPAPPPISGGGYR (SEQ ID NO:132).

Similarly for a collagen-binding peptide, a peptide derived from a phage display library selected for collagen can be generated. The peptide can be synthesized and evaluated for binding to collagen by any of the techniques such as SPR, ELISA, ITC, affinity chromatography, or others known in the art. An example could be a biotin modified peptide sequence (e.g., SILYbiotin) that is incubated on a microplate containing immobilized collagen. A dose response binding curve can be generated using a streptavidin-chromophore to determine the ability of the peptide to bind to collagen.

In one embodiment, the peptides comprise one or more collagen-binding units which binds any one or more of collagen type I, III or IV. In one embodiment, the peptide binds to type I collagen with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM. In one embodiment, the peptide binds to type III collagen with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM. In one embodiment, the peptide binds to type IV collagen with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM. In one embodiment, the peptide binds to type IV collagen with a dissociation constant (Kd) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM.

### ICAM, VCAM and Selectin-Binding Peptides

“ICAM, VCAM and/or selectin binding peptides” are peptides comprising 1 to about 120 amino acids having one or more collagen-binding units (or sequences). As used herein, the term “ICAM, VCAM and/or selectin binding unit” is intended to refer to an amino acid sequence within a peptide which binds to one or more of an ICAM, VCAM and/or selectin receptor. The binding indicates an interaction with an ICAM, VCAM and/or selectin receptor that could include hydrophobic, ionic (charge), and/or Van der Waals interactions, such that the compound binds or interacts favorably with an ICAM, VCAM and/or selectin receptor. This binding (or interaction) is intended to be differentiated from covalent bonds and nonspecific interactions with common functional groups, such that the ICAM, VCAM and/or selectin binding peptide or unit would interact with any species containing that functional group to which the peptide binds on the ICAM, VCAM and/or selectin receptor. In one embodiment, the peptide, or binding unit, binds to an ICAM, VCAM and/or selectin receptor with a dissociation constant (K_{d}) of less than about 1 mM, or less than about 900 μM, or less than about 800 μM, or less than about 700 μM, or less than about 600 μM, or less than about 500 μM, or less than about 400 μM, or less than about 300 μM, or less than about 200 μM, or less than about 100 μM.

Examples of useful peptides include the following peptide sequences (or units), which can bind to selectins: IELLQAR (SEQ ID NO:133), IELLQARGSC (SEQ ID NO:134), IDLMQAR (SEQ ID NO:135), IDLMQARGSC (SEQ ID NO:136), QITWAQLWNMMK (SEQ ID NO:137), QITWAQLWNMMKGSC (SEQ ID NO:138), and combinations thereof. The selectin can be a S-, P- or E-selectin. Various methods for screening peptide sequences for E-selectin-binding affinity (or a E-selectin-binding unit) are routine in the art (see, e.g., Martens, C. L. et al. J. Biol. Chem. 1995, 270(36), 21129-21136; and Koivunen, E. et al. J. Nucl. Med. 1999, 40, 883-888).

Other peptide sequences shown to have E-selectin-binding affinity (or an E-selectin-binding unit) which can be used in bioconjugates and methods disclosed herein include but are not limited to, LRRASLGDGDITWDQLWDLMK (SEQ ID NO:139), HITWDQLWNVMN (SEQ ID NO:140), QITWAQLWNMMK (SEQ ID NO:137), YGNSNITWDQLWSIMNRQTT (SEQ ID NO:141), WTDTHITWDQLWHFMNMGEQ (SEQ ID NO:142), EPWDQITWDQLWIIMNNGDG (SEQ ID NO:143), HITWDQLWLMMS (SEQ ID NO:144), DLTWEGLWILMT (SEQ ID NO:145), RGVWGGLWSMTW (SEQ ID NO:146), DYSWHDLWFMMS (SEQ ID NO:147), KKEDWLALWRIMSVPDEN (SEQ ID NO:148), RNMSWLELWEHMK (SEQ ID NO:149), KEQQWRNLWKMMS (SEQ ID NO:150), SQVTWNDLWSVMNPEVVN (SEQ ID NO:151) and RSLSWLQLWDWMK (SEQ ID NO:152) (see, e.g., Martens, C. L. et al. J. Biol. Chem. 1995, 270(36), 21129-21136), DITWDQLWDLMK (SEQ ID NO:153) (see, e.g., Koivunen, E. et al. J. Nucl. Med. 1999, 40, 883-888), DITWDELWKIMN (SEQ ID NO:154), DYTWFELWDMMQ (SEQ ID NO:155), DMTHDLWLTLMS (SEQ ID NO:156), EITWDQLWEVMN (SEQ ID NO:157), HVSWEQLWDIMN (SEQ ID NO:158), HITWDQLWRIMT (SEQ ID NO:159), DISWDDLWIMIVIN (SEQ ID NO:160), QITWDQLWDLMY (SEQ ID NO:161), RNMSWLELWEHMK (SEQ ID NO:149), AEWTWDQLWHVMNPAESQ (SEQ ID NO:162), HRAEWLALWEQMSP (SEQ ID NO:163), KKEDWLALWRIMSV (SEQ ID NO:164), KRKQWIELWNIMS (SEQ ID NO:165), WKLDTLDMIWQD (SEQ ID NO:166) and HITWDQLWNVMLRRAASLG (SEQ ID NO:167) (see, e.g., Simanek, E. E. Chem. Rev. 1998, 98, 833-862), or combinations thereof, wherein each is hereby incorporated by reference in its entirety.

Various methods for screening peptide sequences for ICAM-binding affinity (or a ICAM-binding unit) are routine in the art (see, e.g., Martens, C. L. et al. J. Biol. Chem. 1995, 270(36), 21129-21136; and Koivunen, E. et al. J. Nucl. Med. 1999, 40, 883-888). Examples of useful peptide sequences that can bind ICAM include the following: NAFKILVVITFGEK (SEQ ID NO:168), NAFKILVVITFGEKGSC (SEQ ID NO:169), ITDGEA (SEQ ID NO:170), ITDGEAGSC (SEQ ID NO:171), DGEATD (SEQ ID NO:172), DGEATDGSC (SEQ ID NO:173), and combinations thereof.

Other peptide sequences shown to have ICAM-binding affinity (or a ICAM-binding unit) which can be used in bioconjugates and methods disclosed herein include but are not limited to, EWCEYLGGYLRYCA (SEQ ID NO:174) (see, e.g., Welply, J. K. et al. Proteins: Structure, Function, and Bioinformatics 1996, 26(3): 262-270), FEGFSFLAFEDFVSSI (SEQ ID NO:175) (see, e.g., US Publication No. WO2014059384), NNQKIVNLKEKVAQLEA (SEQ ID NO:176), NNQKIVNIKEKVAQIEA (SEQ ID NO:177), NNQKLVNIKEKVAQIEA (SEQ ID NO:178), YPASYQR (SEQ ID NO:179), YQATPLP (SEQ ID NO:180), GSLLSAA (SEQ ID NO:181), FSPHSRT (SEQ ID NO:182), YPFLPTA (SEQ ID NO:183) and GCKLCAQ (SEQ ID NO:184) (see, e.g., U.S. Pat. No. 8,926,946), GGTCGGGGTGAGTTTCGTGGTAGGGATAATTCTGTTTGGGTGGTT (SEQ ID NO:185), EWCEYLGGYLRCYA (SEQ ID NO:186) (see, e.g., Koivunen, E. et al. J. Nucl. Med. 1999, 40, 883-888), GRGEFRGRDNSVSVV (SEQ ID NO:187) (see, e.g., CN Publication No. CN1392158), QTSVSPSKVI (SEQ ID NO:188), PSKVILPRGG (SEQ ID NO:189), LPRGGSVLVTG (SEQ ID NO:190), and QTSVSPSKVILPRGGSVLVTG (SEQ ID NO:191) (see, e.g., Tibbetts, S. A. et al. Peptides 21-2000 1161-1167), and combinations thereof, wherein each is hereby incorporated by reference in its entirety.

Various methods for screening peptide sequences for VCAM-binding affinity (or a VCAM-binding unit) are routine in the art (see, e.g., Martens, C. L. et al. J. Biol. Chem. 1995, 270(36), 21129-21136; and Koivunen, E. et al. J. Nucl. Med. 1999, 40, 883-888). Other peptide sequences shown to have VCAM-binding affinity (or a VCAM-binding unit) which can be used in bioconjugates and methods disclosed herein include but are not limited to, YRLAIRLNER (SEQ ID NO:192), YRLAIRLNERRENLRIALRY (SEQ ID NO:193) and RENLRIALRY (SEQ ID NO:194) (see, e.g., EP Publication No. EP1802352), and combinations thereof, which is hereby incorporated by reference in its entirety.

In any of the embodiments described herein, the peptide having a VE-cadherin binding unit, collagen-binding unit, an ICAM-binding unit, a VCAM-binding unit, and/or a selectin-binding unit, comprises any amino acid sequence described in the preceding paragraphs or an amino acid sequence having at least about 80%, or at least about 83%, or at least about 85%, or at least about 90%, or at least about 95%, or at least about 98%, or at least about 100% homology to any of these amino acid sequences. In various embodiments, the peptide components of the bioconjugates described herein can be modified by the inclusion of one or more conservative amino acid substitutions. As is well-known to those skilled in the art, altering any non-critical amino acid of a peptide by conservative substitution should not significantly alter the activity of that peptide because the side-chain of the replacement amino acid should be able to form similar bonds and contacts to the side chain of the amino acid which has been replaced.

Glycans

The bioconjugates of the present disclosure can include a glycan and at least one peptide comprising a VE-Cadherin binding unit. It is contemplated that any glycan can be utilized in the various embodiments described herein, including, but not limited to, alginate, chondroitin, chondroitin sulfate, dermatan, dermatan sulfate, heparan, heparan sulfate, heparin, dextran, dextran sulfate, and hyaluronan, or a derivative thereof. The glycan can be naturally occurring or chemically derivatized, such as, but not limited to, partially N-desulfated derivatives, partially O-desulfated derivatives, and/or partially O-carboxymethylated derivatives.

As used herein, the term “glycan” refers to a compound having a large number of monosaccharides linked glycosidically. In certain embodiments, the glycan is a glycosaminoglycan (GAG), which comprise 2-aminosugars linked in an alternating fashion with uronic acids, and include polymers such as heparin, heparan sulfate, chondroitin, keratin, and dermatan. Accordingly, non-limiting examples of glycans which can be used in the embodiments described herein include alginate, agarose, dextran (Dex), chondroitin, chondroitin sulfate (CS), dermatan, dermatan sulfate (DS), heparan sulfate, heparin (Hep), keratin, keratan sulfate, and hyaluronic acid (HA). In one embodiment, the molecular weight of the glycan is a key parameter in its biological function. In another embodiment, the molecular weight of the glycan is varied to tailor the effects of the bioconjugate (see e.g., Radek, K. A., et al., Wound Repair Regen., 2009, 17: 118-126; and Taylor, K. R., et al., J. Biol. Chem., 2005, 280:5300-5306). In certain embodiments, the glycan molecular weight is about 100 kDa. In certain embodiments, the glycan molecular weight is about 46 kDa. In another embodiment, the glycan is degraded by oxidation and alkaline elimination (see e.g., Fransson, L. A., et al., Eur. J. Biochem., 1980, 106:59-69) to afford degraded glycan having a lower molecular weight (e.g., from about 10 kDa to about 50 kDa). In some embodiments, the glycan is unmodified. In one embodiment, the glycan is heparin. In one embodiment, the glycan is hyaluronan. In one embodiment, the glycan is chondroitin sulfate. In one embodiment, the glycan is dermatan sulfate.

In certain embodiments, the glycan is heparin. Heparin is a highly sulfated glycosaminoglycan, is widely used as an injectable anticoagulant, and has the highest negative charge density of any known biological molecule. Heparin is a naturally occurring anticoagulant produced by basophils and mast cells. Native heparin is a polymer with a molecular weight ranging from 3 to 30 kDa, although the average molecular weight of most commercial heparin preparations is in the range of 12 to 15 kDa. Heparin is a member of the glycosaminoglycan family of carbohydrates (which includes the closely related molecule heparan sulfate) and consists of a variably sulfated repeating disaccharide unit. The most common disaccharide unit is composed of a 2-O-sulfated iduronic acid and 6-O-sulfated, N-sulfated glucosamine, IdoA(2S)-GlcNS(6S). Various molecular weights for the heparin can be used in the bioconjugates described herein, such as from a single disaccharide unit of about 650-700 Da to about 50 kDa. In some embodiments, the heparin is from about 10 to about 20 kDa. In some embodiments, the heparin is up to about 15, or about 16, or about 17, or about 18, or about 19, or about 20 kDa. In certain embodiments, the heparin may be oxidized under conditions that do not cleave one or more of the saccharide rings (see, e.g., Wang, et al. Biomacromolecules 2013, 14(7):2427-2432). In one embodiment, the heparin may include heparin derivatives, such as, but not limited to partially N- and/or partially O-desulfated heparin derivatives, partially O-carboxymethylated heparin derivatives, or a combination thereof. In certain embodiments, the heparin is non-oxidized heparin (i.e., does not contain oxidatively cleaved saccharide rings) and does not contain aldehyde functional groups. Heparin derivatives and/or methods for providing heparin derivatives, such as partially N-desulfated heparin and/or partially O-desulfated heparin (i.e., 2-0 and/or 6-O-desulfated heparin) are known in the art (see, e.g., Kariya et al., J. Biol. Chem., 2000, 275:25949-5958; Lapierre, et al. Glycobiology, 1996, 6(3):355-366). It is also contemplated that partially O-carboxymethylated heparin (or any glycan) derivatives, such as those which could be prepared according to Prestwich, et al. (US 2012/0142907; US 2010/0330143), can be used to provide the bioconjugates disclosed herein.

Bioconjugates

The peptide(s) can be bonded to the glycan directly or via a linker. As used herein, the terms “bound”, “bonded” and “covalently bonded” can be used interchangeably, and refer to the sharing of one or more pairs of electrons by two atoms. In one embodiment, the peptide is bonded to the glycan. In one embodiment, the peptide is covalently bonded to the glycan, with or without a linker. In one embodiment the peptide is covalently bonded to the glycan via a linker. In one embodiment the peptide is directly bonded to the glycan.

In some embodiments, the linker can be any suitable bifunctional linker, e.g., N-[β-maleimidopropionic acid]hydrazide (BMPH), 3-(2-pyridyldithio)propionyl hydrazide (PDPH), and the like. In any of the various embodiments described herein, the sequence of the peptide may be modified to include a glycine-cysteine (GC) attached to the C-terminus of the peptide and/or a glycine-cysteine-glycine (GCG) attached to the N-terminus to provide an attachment point for a glycan or a glycan-linker conjugate. In certain embodiments, the linker is N-[β-maleimidopropionic acid]hydrazide (BMPH). In certain embodiments, the linker is 3-(2-pyridyldithio)propionyl hydrazide (PDPH). In some embodiments, the peptide to linker ratio is from about 1:1 to about 5:1. In certain embodiments, the peptide to linker ratio is from about 1:1 to about 10:1. In certain embodiments, the peptide to linker ratio is from about 1:1 to about 2:1, or from about 1:1 to about 3:1, or from about 1:1 to about 4:1, or from about 1:1 to about 5:1, or from about 1:1 to about 6:1, or from about 1:1 to about 7:1, or from about 1:1 to about 8:1, or from about 1:1 to about 9:1. In one embodiment, the peptide to linker ratio is about 1:1. In one embodiment, the peptide to linker ratio is about 2:1. In one embodiment, the peptide to linker ratio is about 3:1. In one embodiment, the peptide to linker ratio is about 4:1. In one embodiment, the peptide to linker ratio is about 5:1. In one embodiment, the peptide to linker ratio is about 6:1. In one embodiment, the peptide to linker ratio is about 7:1. In one embodiment, the peptide to linker ratio is about 8:1. In one embodiment, the peptide to linker ratio is about 9:1. In one embodiment, the peptide to linker ratio is about 10:1.

Depending on the desired properties of the bioconjugate, the total number of peptides bonded to the glycan can be varied. In certain embodiments, the total number of peptides present in the bioconjugate is from about 1 or 2 to about 160, or from about 10 to about 160, or from about 20 to about 160, or from about 30 to about 160, or from about 40 to about 160, or from about 40 to about 150, or from about 40 to about 140, or from about 10 to about 120, or from about 20 to about 110, or from about 20 to about 100, or from about 20 to about 90, or from about 30 to about 90, or from about 40 to about 90, or from about 50 to about 90, or from about 50 to about 80, or from about 60 to about 80, or about 10, or about 20, or about 30, or about 40, or about 50, or about 60, or about 70, or about 80, or about 90, or about 100, or about 110, or about 120. In certain embodiments, the bioconjugate comprises about 70 peptides. In certain embodiments, the bioconjugate comprises from about 50 to about 80, or from about 55 to about 75, or about 55, or about 60, or about 65, or about 70, or about 75 peptides. In certain embodiments, the bioconjugate comprises less than about 50 peptides. In various embodiments, the bioconjugate comprises from about 5 to about 40 peptides. In some embodiments, the bioconjugate comprises from about 10 to about 40 peptides. In certain embodiments, the bioconjugate comprises from about 5 to about 20 peptides. In various embodiments, the bioconjugate comprises from about 4 to about 18 peptides. In certain embodiments, the bioconjugate comprises less than about 20 peptides. In certain embodiments, the bioconjugate comprises less than about 18 peptides. In certain embodiments, the bioconjugate comprises less than about 15 peptides. In certain embodiments, the bioconjugate comprises less than about 10 peptides. In certain embodiments, the bioconjugate comprises about 20 peptides. In certain embodiments, the bioconjugate comprises about 40 peptides. In certain embodiments, the bioconjugate comprises about 18 peptides. In certain embodiments, the bioconjugate comprises from about 5 to about 40, or from about 10 to about 40, or from about 5 to about 20, or from about 4 to about 18, or about 10, or about 11, or about 18, or about 20 peptides.

The peptides can be bound to the glycan via the C-terminus, the N-terminus or a side chain of an amino acid in the peptide. In certain embodiments, the peptide has a free N-terminus. In certain embodiments, the peptide does not have a free N-terminus, where an additional chemical moiety is bound thereto, including, but not limited to, a peptide, a protecting group, a label, etc.

In one embodiment, provided herein is a bioconjugate comprising a glycan and at least one peptide comprising an amino acid sequence PSLRPAPPPISGGGYR (SEQ ID NO:1), or an amino acid sequence having one, two, or three amino acid addition, deletion and/or substitution(s) therefrom. In one embodiment, provided herein is a bioconjugate comprising a glycan and at least one peptide comprising an amino acid sequence GHRPLDKKREEAPSLRPA (SEQ ID NO:2), or an amino acid sequence having one, two, or three amino acid addition, deletion and/or substitution(s) therefrom. In another embodiment, provided herein is a bioconjugate comprising a glycan and at least one peptide comprising an amino acid sequence GHRPLDKKREEAPSLRPAPPPISGGGYR (SEQ ID NO:3), or an amino acid sequence having one, two, or three amino acid addition, deletion and/or substitution therefrom.

In one embodiment, provided herein is a bioconjugate comprising a glycan and at least one peptide comprising an amino acid sequence PSLRPAPPPISGGGYRGSG (SEQ ID NO:8), or an amino acid sequence having one, two, or three amino acid addition, deletion and/or substitution(s) therefrom. In one embodiment, provided herein is a bioconjugate comprising a glycan and at least one peptide comprising an amino acid sequence GHRPLDKKREEAPSLRPAGSG (SEQ ID NO:33), or an amino acid sequence having one, two, or three amino acid addition, deletion and/or substitution(s) therefrom. In another embodiment, provided herein is a bioconjugate comprising a glycan and at least one peptide comprising an amino acid sequence GHRPLDKKREEAPSLRPAPPPISGGGYRGSG (SEQ ID NO:14), or an amino acid sequence having one, two, or three amino acid addition, deletion and/or substitution therefrom.

In one embodiment, provided herein is a bioconjugate comprising a glycan and at least one peptide comprising an amino acid sequence RYGGGSIPPPAPRLSP (SEQ ID NO:195), or an amino acid sequence having one, two, or three amino acid addition, deletion and/or substitution(s) therefrom. In one embodiment, provided herein is a bioconjugate comprising a glycan and at least one peptide comprising an amino acid sequence APRLSPAEERKKDLPRHG (SEQ ID NO:196), or an amino acid sequence having one, two, or three amino acid addition, deletion and/or substitution(s) therefrom. In another embodiment, provided herein is a bioconjugate comprising a glycan and at least one peptide comprising an amino acid sequence RYGGGSIPPPAPRLSPAEERKKDLPRHG (SEQ ID NO:197), or an amino acid sequence having one, two, or three amino acid addition, deletion and/or substitution therefrom.

In one embodiment, provided herein is a bioconjugate comprising comprising a glycan and at least one peptide bonded thereto comprising the sequence GHRPLDKKREEAPSLRPA (SEQ ID NO:2). In one embodiment, the bioconjugate comprises from 1 to about 100 peptides per glycan. In one embodiment, provided herein is a bioconjugate comprising a glycan and from about 50 to about 80 peptides bonded thereto comprising the sequence GHRPLDKKREEAPSLRPA (SEQ ID NO:2). In one embodiment, provided herein is a bioconjugate comprising a glycan and from about 60 to about 70 peptides bonded thereto comprising the sequence GHRPLDKKREEAPSLRPA (SEQ ID NO:2). In another embodiment, provided herein is a bioconjugate comprising hyaluronan and from about 50 to about 80 peptides bonded thereto comprising the sequence GHRPLDKKREEAPSLRPA (SEQ ID NO:2) or GHRPLDKKREEAPSLRPAGSG (SEQ ID NO:33). In one embodiment, provided herein is a bioconjugate comprising hyaluronan and from about 60 to about 70 peptides bonded thereto comprising the sequence GHRPLDKKREEAPSLRPA (SEQ ID NO:2) or GHRPLDKKREEAPSLRPAGSG (SEQ ID NO:33). In certain embodiments, the peptides are bound to the glycan (e.g., hyaluronan, heparin, dermatan sulfate, etc.) via a hydrazide-carbonyl bond.

In another embodiment, provided herein is a bioconjugate comprising a glycan and at least one peptide bonded thereto comprising the sequence CRVDAE-Ahx-RVDAEC (SEQ ID NO:12), wherein the peptide is cyclized at the cysteines and Ahx is 6-aminohexanoic acid, or a sequence having at least about 80% sequence identity, or at least about 83% sequence identity, or at least about 85% sequence identity, or at least about 90% sequence identity, or at least about 95% sequence identity, or at least about 98% sequence identity, or at least about 99% sequence identity thereto, provided the sequence is capable of binding to VE-cadherin. In certain embodiments, provided herein is a bioconjugate comprising a glycan and at least one peptide bonded thereto comprising the sequence CRVDAE-Ahx-RVDAEC (SEQ ID NO:12) or CRVDAE-Ahx-RVDAECGSG (SEQ ID NO:13), wherein the peptide is cyclized at the cysteines and Ahx is 6-aminohexanoic acid, or an amino acid sequence having one, two, or three amino acid addition, deletion and/or substitution therefrom.

In any of the embodiments described herein, the number of peptides per glycan is an average, where certain bioconjugates in a composition may have more peptides per glycan and certain bioconjugates have less peptides per glycan. Accordingly, in certain embodiments, the number of peptides as described herein is an average in a composition of bioconjugates. For example, in certain embodiments, the bioconjugates are a composition where the average number of peptides per glycan is about 5. In certain embodiments, the average number of peptides per glycan is about 6, or about 7, or about 8, or about 9, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 80. In certain embodiments, the average number of peptides per glycan is about 3. In certain embodiments, the average number of peptides per glycan is about 4. In certain embodiments, the average number of peptides per glycan is about 30. In certain embodiments, the average number of peptides per glycan is about 60. In certain embodiments, the average number of peptides per glycan is about 70. In certain embodiments, the number of peptides per glycan may be described as a “percent (%) functionalization” based on the percent of disaccharide units which are functionalized with peptide on the glycan backbone. For example, the total number of available disaccharide units present on the glycan can be calculated by dividing the molecular weight (or the average molecular weight) of a single disaccharide unit (e.g., about 550-800 Da, or from about 650-750 Da) by the molecular weight of the glycan (e.g., about 25 kDa up to about 70 kDa, or even about 100 kDa). For example, in some embodiments, the number of available disaccharide units present on the glycan is from about 10 to about 80, or from about 10 to about 70, or from about 15 to about 70, or from about 20 to about 70, or from about 30 to about 70, or from about 35 to about 70, or from about 40 to about 70, or from about 10 to about 50, or from about 20 to about 50, or from about 25 to about 50, or from about 10 to about 30, or from about 15 to about 30, or from about 20 to about 30, or about 15, or about 20, or about 25, or about 30, or about 35, or about 40, or about 45, or about 50, or about 55, or about 60, or about 65, or about 70.

Therefore, in certain embodiments, the glycan comprises from about 1 to about 50, or from about 10 to about 50, or from about 5 to about 30% functionalization, or about 25% functionalization, wherein the percent (%) functionalization is determined by a percent of disaccharide units on the glycan which are functionalized with peptide. In some embodiments, the percent (%) functionalization of the glycan is from about 1% to about 50%, or from about 3% to about 40%, or from about 10% to about 40%, or from about 20% to about 40%, or from about 5% to about 30%, or from about 10% to about 20%, or about 1%, or about 2%, or about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%.

In one embodiment, provided herein is a bioconjugate comprising a peptide comprising GHRPLDKKREEAPSLRPA (SEQ ID NO:2), wherein the percent (%) functionalization of the glycan is from about 1% to about 75%, or from about 1% to about 60%, or from about 1% to about 50%, or from about 5% to about 40%, or from about 10% to about 40%, or from about 20% to about 40%, or from about 5% to about 30%, or from about 10% to about 20%, or about 1%, or about 2%, or about 5%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%.

In one embodiment, provided herein is a bioconjugate comprising a glycan and at least one peptide bound thereto, wherein the peptide comprises the sequence GHRPLDKKREEAPSLRPA (SEQ ID NO:2), wherein the percent (%) functionalization of the glycan with peptide is from about 1% to about 60%. In one embodiment, provided herein is a bioconjugate comprising a glycan and at least one peptide bound thereto, wherein the peptide comprises the sequence GHRPLDKKREEAPSLRPA (SEQ ID NO:2), wherein the percent (%) functionalization of the glycan is from about 20% to about 40%. In one embodiment, provided herein is a bioconjugate comprising a glycan and at least one peptide bound thereto, wherein the peptide comprises the sequence GHRPLDKKREEAPSLRPA (SEQ ID NO:2), wherein the percent (%) functionalization of the glycan is from about 25% to about 35%. In one embodiment, provided herein is a bioconjugate comprising a glycan and at least one peptide bound thereto, wherein the peptide comprises the sequence GHRPLDKKREEAPSLRPA (SEQ ID NO:2), wherein the percent (%) functionalization of the glycan is about 30%.

In one embodiment, provided herein is a bioconjugate comprising hyaluronan and at least one peptide bound thereto, wherein the peptide comprises the sequence GHRPLDKKREEAPSLRPA (SEQ ID NO:2), wherein the percent (%) functionalization of the hyaluronan with peptide is from about 1% to about 60%. In one embodiment, provided herein is a bioconjugate comprising hyaluronan and at least one peptide bound thereto, wherein the peptide comprises the sequence GHRPLDKKREEAPSLRPA (SEQ ID NO:2), wherein the percent (%) functionalization of the hyaluronan is from about 20% to about 40%. In one embodiment, provided herein is a bioconjugate comprising hyaluronan and at least one peptide bound thereto, wherein the peptide comprises the sequence GHRPLDKKREEAPSLRPA (SEQ ID NO:2), wherein the percent (%) functionalization of the hyaluronan is from about 25% to about 35%. In one embodiment, provided herein is a bioconjugate comprising hyaluronan and at least one peptide bound thereto, wherein the peptide comprises the sequence GHRPLDKKREEAPSLRPA (SEQ ID NO:2), wherein the percent (%) functionalization of the hyaluronan is about 30%.

Therefore, in some embodiments, peptides are bound to glycans, such as dermatan sulfate, by utilizing oxidation chemistry to cleave one or more of the saccharide ring within the glycan backbone in order to provide aldehyde binding sites on the glycan. The aldehyde binding sites are then used to conjugate the peptides (e.g., via a —C(O)—NH—N═C bond).

In some embodiments, the peptides can be covalently bound to glycan via a —C(O)—NH—NH—C(O)— (i.e. a hydrazide-carbonyl) linkage. Here, the peptides are bound to the glycan via a hydrazide-carbonyl linkage, where a carbonyl group of the hydrazide-carbonyl is an exocyclic carbonyl group present on the glycan. The exocyclic carbonyl group may be present on the native glycan, or alternatively, the glycan can be modified to include such a functional group. Such methods are further detailed below. It is contemplated that the beneficial effects exhibited by the bioconjugates as disclosed herein (such as increased binding affinity) is at least partially due to the glycan not containing oxidatively cleaved saccharide rings.

Accordingly, in certain embodiments, the peptides as described herein further comprise a hydrazide moiety for conjugation to the peptide. The hydrazide group can be bound to the peptide(s) at any suitable point of attachment, such as for example, the C-terminus, the N-terminus or via a side chain on an amino acid. For example, when a peptide is bound to the glycan via a side chain of an amino acid of the peptide, the side chain is glutamic acid or aspartic acid. The hydrazide can be formed between a hydrazine (—NHNH₂) bound to a carbonyl group present on an amino acid in the peptide sequence (e.g., a C-terminal carbonyl group) or to a spacer, if present.

In certain embodiments, the peptide(s) are bonded to the glycan (or the linker, if present) via a spacer. As used herein, the term “spacer” is intended to refer to an optional portion of the bioconjugate which links the peptide (or binding unit) to either the linker, when present, or the glycan (can be bound directly). In any of the embodiments described herein, any one or more of the peptides may have a linear or branched spacer sequence comprising from 1 to about 15 amino acids. In one embodiment, the spacer comprises one or more, or from 1 to 10, or from 1 to 5, or from 1 to 3, amino acids. It is contemplated that any amino acid, natural or unnatural, can be used in the spacer sequence, provided that the spacer sequence does not significantly interfere with the intended binding of the peptide. The amino acids are, in some instances, non-polar amino acids, such as alanine, cysteine, glycine, isoleucine, leucine, methionine, phenylalanine, proline, tryptophan, tyrosine and valine. In certain embodiments, the amino acids are selected from the group consisting of glycine, alanine, arginine and serine.

Exemplary spacers include, but are not limited to, short sequences comprising from one to five glycine units (e.g., G, GG, GGG, GGGG, or GGGGG), optionally comprising cysteine (e.g., GC, GCG, GSGC, or GGC) and/or serine (e.g., GSG, SGG, or GSGSG), or from one to five arginine units (e.g., R, RR, RRR, etc.). In one embodiment, the spacer is selected from the group consisting of glycine (G), glycine-glycine (GG), and glycine-serine-glycine(GSG). In certain embodiments, the spacer comprises from 1 to 15 amino acids, or from 5 to 10, or 5 amino acids. In certain embodiments, the amino acids of the spacer comprise glycine, serine and arginine, or combinations thereof. In certain embodiments, the spacer is a sequence of from 1 to 15 amino acids, or from 5 to 10, or 5 amino acids comprised of glycine, serine and arginine. The spacer may also comprise non-amino acid moieties, such as polyethylene glycol (PEG), 6-aminohexanoic acid, succinic acid, or combinations thereof, with or without an additional amino acid spacer.

In certain embodiments, the spacer comprises more than one binding site (where the spacer may be linear or branched) such that more than one peptide sequence can be bound thereto, thus creating a branched construct. In addition, since the peptide can be bound to the glycan via a terminal or non-terminal amino acid moiety, the peptide will be branched when bound to the glycan via a non-terminal amino acid moiety. The binding sites on the spacer can be the same or different, and can be any suitable binding site, such as an amine or carboxylic acid moiety, such that a desired peptide sequence can be bound thereto (e.g. via an amide bond). Thus in certain embodiments, the spacer contains one or more lysine, glutamic acid or aspartic acid residues. In certain embodiments, the spacer comprises from 2 to 6 amino acids, or 3 or 4 amino acids. In certain embodiments, the spacer comprises one or more amino acid sequences of the formula KXX, where each X is independently a natural or unnatural amino acid. Specific examples of spacers which can be used alone or in combination to make branched constructs include, but are not limited to, KRR, KKK, (K)ₙ-GSG, and (KRR)ₙ-KGSG, where n is 0 to 5, or 1, 2, 3, 4, or 5.

Such constructs can provide peptides having more than one unit of the formula PnL, where at least one P is a VE-cadherin binding unit, L is a spacer and n is an integer from 2 to about 10, or from 2 to 8, or from 2 to 6, or from 2 to 5, or from 2 to 4, or 2, or 3, or 4, or 5, or 6, or 7, or 8, or 9, or 10 For example, the spacer L can be an amino acid sequence such as KGSG (SEQ ID NO:198), KKGSG (SEQ ID NO:199), K_{K}KGSG (SEQ ID NO:200), or KKKGSG (SEQ ID NO:201), etc., where peptides can be bound to the N-terminus and the side chain nitrogen, providing 2, 3, and 4 binding sites, respectively. The branched spacers may or may not include the additional linear sequence -GSG. For example, the spacer L can be an amino acid sequence such as KK, K₂K, or KKK, etc., where peptides can be bound to the N-terminus and the side chain nitrogen, providing 3 and 4 binding sites. A schematic of these spacers bound to peptides is shown in the table below.

| | Number of | |
|---|---|---|
| | peptides (i.e., | |
| Spacer | binding sites) | Structure of Spacer |
| KGSG (SEQ ID NO: 198) | 2 | 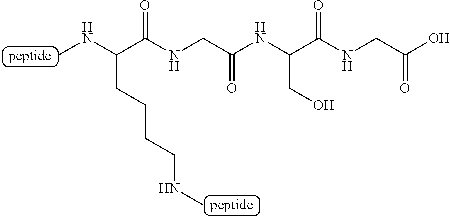 [SMILES: CNCCCCC(NC)C(=O)NCC(=O)NC(CO)C(=O)NCC(=O)O] |
| KKGSG (SEQ ID NO: 199) | 3 | 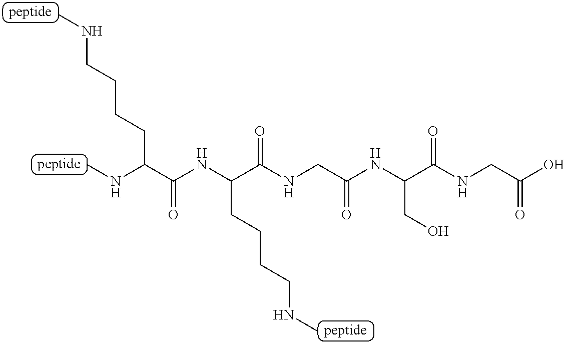 [SMILES: CNCCCCC(NC)C(=O)NC(CCCCNC)C(=O)NCC(=O)NC(CO)C(=O)NCC(=O)O] |
| KKKGSG (SEQ ID NO: 201) | 4 | 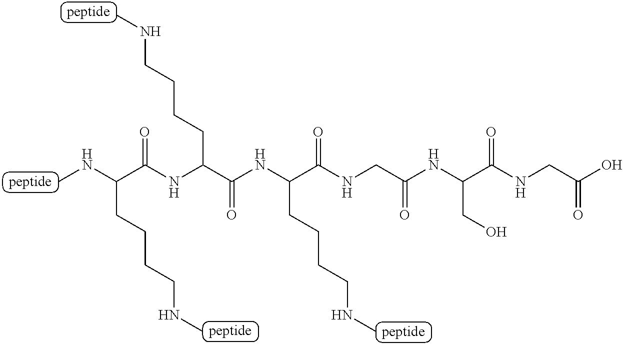 [SMILES: CNCCCCC(NC)C(=O)NC(CCCCNC)C(=O)NC(CCCCNC)C(=O)NCC(=O)NC(CO)C(=O)NCC(=O)O] |
| K₂KGSG (SEQ ID NO: 200) | 4 | 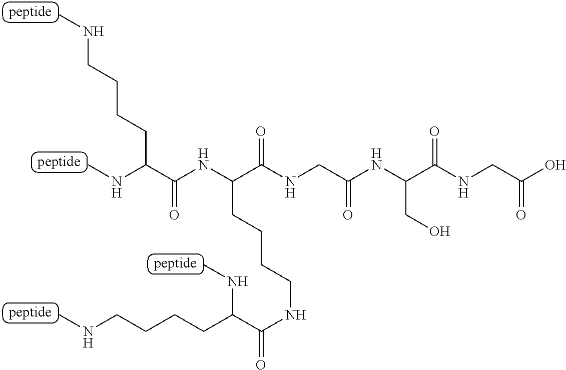 [SMILES: CNCCCCC(NC)C(=O)NCCCCC(NC(=O)C(CCCCNC)NC)C(=O)NCC(=O)NC(CO)C(=O)NCC(=O)O] |
| KKK | 4 | 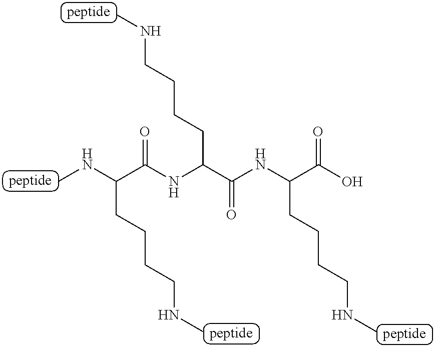 |
| K₂K | 4 | 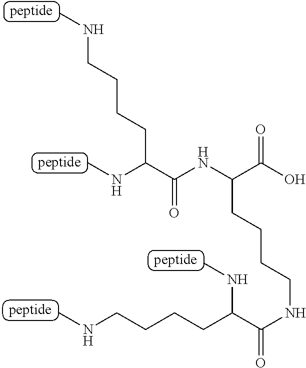 [SMILES: CNCCCCC(NC)C(=O)NCCCCC(NC(=O)C(CCCCNC)NC)C(=O)O] |

In any of the bioconjugates described herein, any one or more peptides may comprise at least one collagen-binding unit, selectin-binding unit, ICAM-binding unit and/or VCAM-binding unit. It is contemplated that bioconjugates having peptides comprising both a VE-cadherin binding unit in combination with a collagen-binding unit may be particularly useful in stabilizing endothelial cell-cell junctions.

Also provided herein are compositions comprising a VE-cadherin binding bioconjugate as described herein, in combination with one or more bioconjugates selected from the group consisting of:
a) a bioconjugate comprising a glycan and at least one peptide comprising a collagen-binding unit;
b) a bioconjugate comprising a glycan and at least one peptide comprising a ICAM-binding unit;
c) a bioconjugate comprising a glycan and at least one peptide comprising a VCAM-binding unit; and
d) a bioconjugate comprising a glycan and at least one peptide comprising a selectin-binding unit.

It is contemplated that compositions comprising a VE-cadherin binding bioconjugate as described herein, in combination with a bioconjugate comprising a glycan and at least one peptide comprising a collagen-binding unit may be particularly useful in the methods described below.

### 3. SYNTHESIS OF BIOCONJUGATES

The peptides used in the method described herein (i.e., the collagen-binding peptide) may be purchased from a commercial source or partially or fully synthesized using methods well known in the art (e.g., chemical and/or biotechnological methods). In certain embodiments, the peptides are synthesized according to solid phase peptide synthesis protocols that are well known in the art. In another embodiment, the peptide is synthesized on a solid support according to the well-known Fmoc protocol, cleaved from the support with trifluoroacetic acid and purified by chromatography according to methods known to persons skilled in the art. In certain embodiments, the peptide is synthesized utilizing the methods of biotechnology that are well known to persons skilled in the art. In one embodiment, a DNA sequence that encodes the amino acid sequence information for the desired peptide is ligated by recombinant DNA techniques known to persons skilled in the art into an expression plasmid (for example, a plasmid that incorporates an affinity tag for affinity purification of the peptide), the plasmid is transfected into a host organism for expression, and the peptide is then isolated from the host organism or the growth medium, e.g., by affinity purification. Recombinant DNA technology methods are described in Sambrook et al., “Molecular Cloning: A Laboratory Manual”, 3rd Edition, Cold Spring Harbor Laboratory Press, (2001), incorporated herein by reference, and are well-known to the skilled artisan.

In certain embodiments, the peptides are covalently bonded to the glycan directly (i.e., without a linker). In such embodiments, the bioconjugates may be formed by covalently bonding the peptides to the glycan through the formation of one or more amide, ester or imino bonds between an acid, aldehyde, hydroxy, amino, or hydrazo group on the glycan. All of these methods are known in the art. See, e.g., Hermanson G. T., Bioconjugate Techniques, Academic Press, pp. 169-186 (1996), incorporated herein by reference. As shown in Scheme 1, the glycan (e.g., chondroitin sulfate “CS”) can be oxidized using a periodate reagent, such as sodium periodate, to provide aldehyde functional groups on the glycan (e.g., “ox-CS”) for covalently bonding the peptides to the glycan. In such embodiments, the peptides may be covalently bonded to a glycan by reacting a free amino group of the peptide with an aldehyde functional groups of the oxidized glycan, e.g., in the presence of a reducing agent, utilizing methods known in the art.

In embodiments where the peptides are covalently bonded to the glycan via a linker, the oxidized glycan (e.g., “ox-CS”) can be reacted with a linker (e.g., any suitable bifunctional liker, such as 3-(2-pyridyldithio)propionyl hydrazide (PDPH) or N-[β-maleimidopropionic acid]hydrazide (BMPH)) prior to contacting with the peptides. The linker typically comprises about 1 to about 30 carbon atoms, or about 2 to about 20 carbon atoms. Lower molecular weight linkers (i.e., those having an approximate molecular weight of about 20 to about 500) are typically employed. In addition, structural modifications of the linker are contemplated. For example, amino acids may be included in the linker, including but not limited to, naturally occurring amino acids as well as those available from conventional synthetic methods, such as beta, gamma, and longer chain amino acids.

As shown in Scheme 1, in one embodiment, the peptides are covalently bonded to the glycan (e.g., chondroitin sulfate “CS”) by reacting an aldehyde function of the oxidized glycan (e.g., “ox-CS”) with N-[β-maleimidopropionic acid]hydrazide (BMPH) to form an glycan intermediate (e.g., “BMPH-CS”) and further reacting the glycan intermediate with peptides containing at least one free thiol group (i.e., —SH group) to yield the bioconjugate. In yet another embodiment, the sequence of the peptides may be modified to include an amino acid residue or residues that act as a spacer between the HA- or Collagen-binding peptide sequence and a terminating cysteine (C). For example a glycine-cysteine (GC) or a glycine-glycine-glycine-cysteine (GGGC) (SEQ ID NO:202) or glycine-serine-glycine-cysteine (GSGC) (SEQ ID NO:203) segment may be added to provide an attachment point for the glycan intermediate.

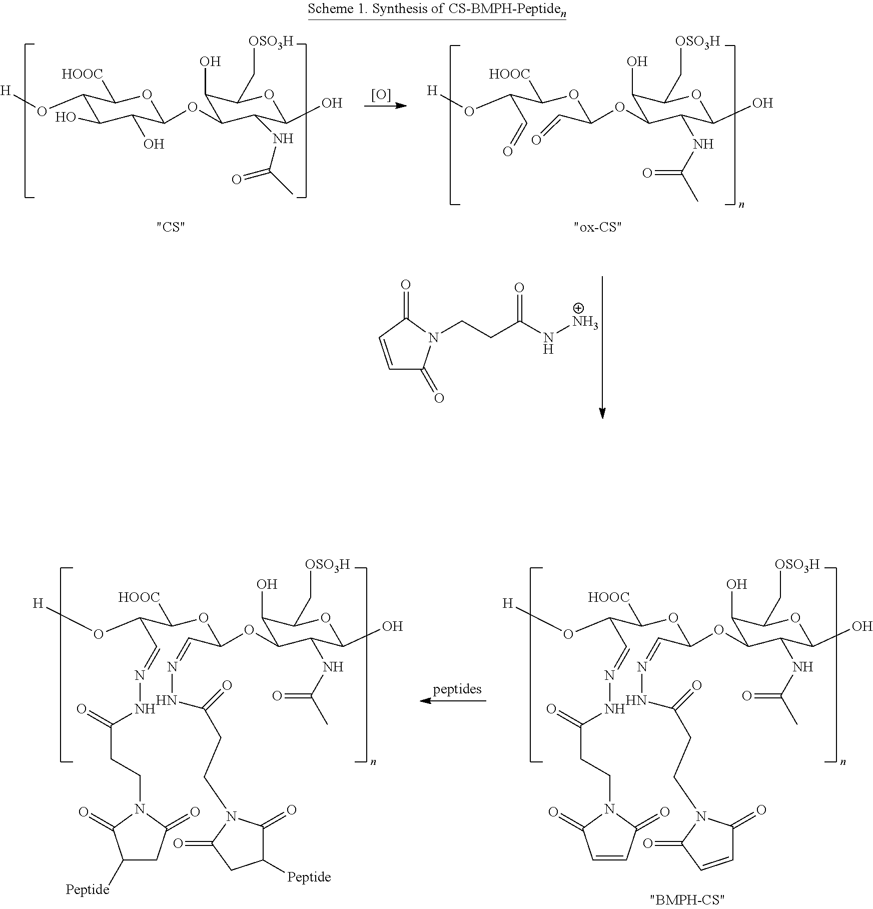

Another example is illustrated in Scheme 2, wherein the peptides as described herein can be covalently bound to the glycan (e.g., heparin) 1A through a carboxylic acid moiety to provide a bioconjugate 1B as disclosed herein. As is typical in peptide coupling reactions, an activating agent may be used to facilitate the reaction. Suitable coupling agents (or activating agents) are known in the art and include for example, carbodiimides (e.g., N,N′-dicyclohexylcarbodiimide (DCC), N,N′-dicyclopentylcarbodiimide, N,N′-diisopropylcarbodiimide (DIC), 1-ethyl-3-(3-dimethylaminopropyl)carbodiimide (EDC), N-t-butyl-N-methylcarbodiimide (BMC), N-t-butyl-N-ethylcarbodiimide (BEC), 1,3-bis(2,2-dimethyl-1,3-dioxolan-4-ylmethyl)carbodiimide (BDDC), etc.), anhydrides (e.g., symmetric, mixed, or cyclic anhydrides), activated esters (e.g., phenyl activated ester derivatives, p-hydroxamic activated ester, hexafluoroacetone (HFA), etc.), acylazoles (acylimidazoles using CDI, acylbenzotriazoles, etc.), acyl azides, acid halides, phosphonium salts (HOBt, PyBOP, HOAt, etc.), aminium/uronium salts (e.g., tetramethyl aminium salts, bispyrrolidino aminium salts, bispiperidino aminium salts, imidazolium uronium salts, pyrimidinium uronium salts, uronium salts derived from N,N,N′-trimethyl-N′-phenylurea, morpholino-based aminium/uronium coupling reagents, antimoniate uronium salts, etc.), organophosphorus reagents (e.g., phosphinic and phosphoric acid derivatives), organosulfur reagents (e.g., sulfonic acid derivatives), triazine coupling reagents (e.g., 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate, etc.), pyridinium coupling reagents (e.g., Mukaiyama's reagent, pyridinium tetrafluoroborate coupling reagents, etc.), polymer-supported reagents (e.g., polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ, etc.), and the like (see, e.g., El-Faham, et al. Chem. Rev., 2011, 111(11): 6557-6602; Han, et al. Tetrahedron, 2004, 60:2447-2467).

In one embodiment, the peptide coupling reaction proceeds via an activated glycan intermediate by reacting a carboxylic acid moiety of the glycan with a coupling agent (e.g., a carbodiimide reagent, such as but not limited to, N,N′-dicyclohexylcarbodiimide (DCC), N,N′-diisopropylcarbodiimide (DIC), 1-ethyl-3-(3-dimethylaminopropyl)carbodiimide (EDC), etc.) to form an O-acylisourea intermediate. Such carbodiimide chemistry is well known in the art and suitable coupling agents can be purchased from commercial sources. Contacting the O-acylisourea intermediate with the desired peptide yields the bioconjugate. The glycan can be contacted with activating agent prior to, or in the presence of, the peptide. In some embodiments, the reaction is carried out in the presence of N-hydroxysuccinimide (NETS) or derivatives thereof. In certain embodiments, the peptide sequence can comprise a reactive moiety (e.g., a hydrazide functional group) to aid in the coupling reaction with the glycan, or O-acylisourea intermediate thereof. In some embodiments, the peptide sequence includes one or more amino acid residues that act as a spacer between the binding unit and the terminal amino acid (e.g., a terminating glycine) or reactive moiety (i.e., hydrazide functional group). For example, a serine-glycine (SG), glycine-serine-glycine (GSG) or glycine-serine-glycine-serine-glycine (GSGSG) spacer may be added to provide an attachment point for the glycan. In addition, in certain instances where one or more amino acids in the peptides contain reactive functional groups (e.g., carboxylic acid side chains), standard protecting group chemistry may be used to protect one or more side chains facilitate the coupling reaction. In addition, non-amino acid spacers may also be employed alone, or in combination with amino acid spacers (e.g., aminohexanoic acid).

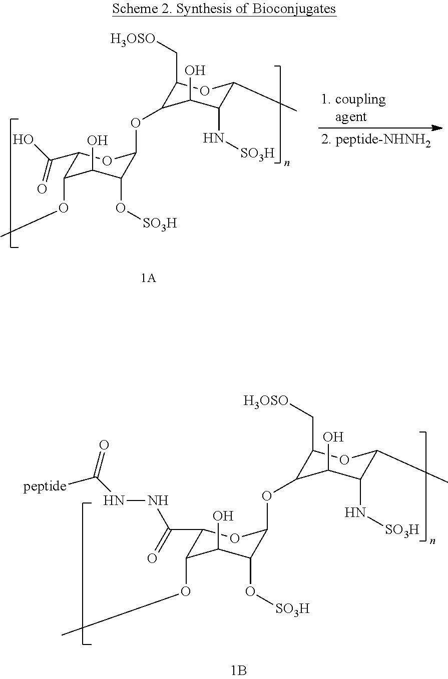

In certain embodiments, the bioconjugates are derived from modified glycan derivatives (e.g., heparin) (Scheme 3). Various glycan derivatives suitable for use in the bioconjugates described herein are known in the art, such as partially N-desulfated heparin and partially 0-desulfated heparin (i.e., 2-0 and/or 6-O-desulfated heparin, see, e.g., Kariya et al., J. Biol. Chem., 2000, 275:25949-5958; Lapierre, et al. Glycobiology, 1996, 6(3):355-366). Exemplary methods are shown below in Scheme 3. As shown in Scheme 3, glycan (e.g., heparin) 1A can be reacted with a suitable desulfating agent, such as for example, a base (e.g., NaOH) or a silylating reagent (e.g., N,O-bis(trimethylsilyl)acetamide (BTSA), N-methyl-N-(trimethylsilyl)trifluoro acetamide (MTSTFA), etc.) to provide one or more desulfated glycan derivative(s) 2A. As is apparent to one of skill in the art, the glycan derivative 2A can be tailored depending on the reagents and reaction conditions employed, such that partial, complete or a mixture of desulfated glycan derivative(s) 2A can be obtained. The desulfated glycan derivative(s) 2A can then be reacted with peptide, optionally in the presence of a coupling agent, as described above for Scheme 2, under typical peptide coupling reaction conditions to provide bioconjugate 2B. In addition, as shown in Scheme 3, glycan derivatives having at least one hydroxyl group (e.g., 6-O-desulfated heparin) can be converted to an O-carboxymethylated glycan derivative(s) (e.g., 6-O-carboxymethylated heparin) 2C (see, e.g., Prestwich, et al. in US 2012/0142907 and US 2010/0330143). Reaction of 2C with peptide, optionally in the presence of a coupling agent as described above for Scheme 2 under typical peptide coupling reaction conditions can provide bioconjugates 2D and/or 2E.

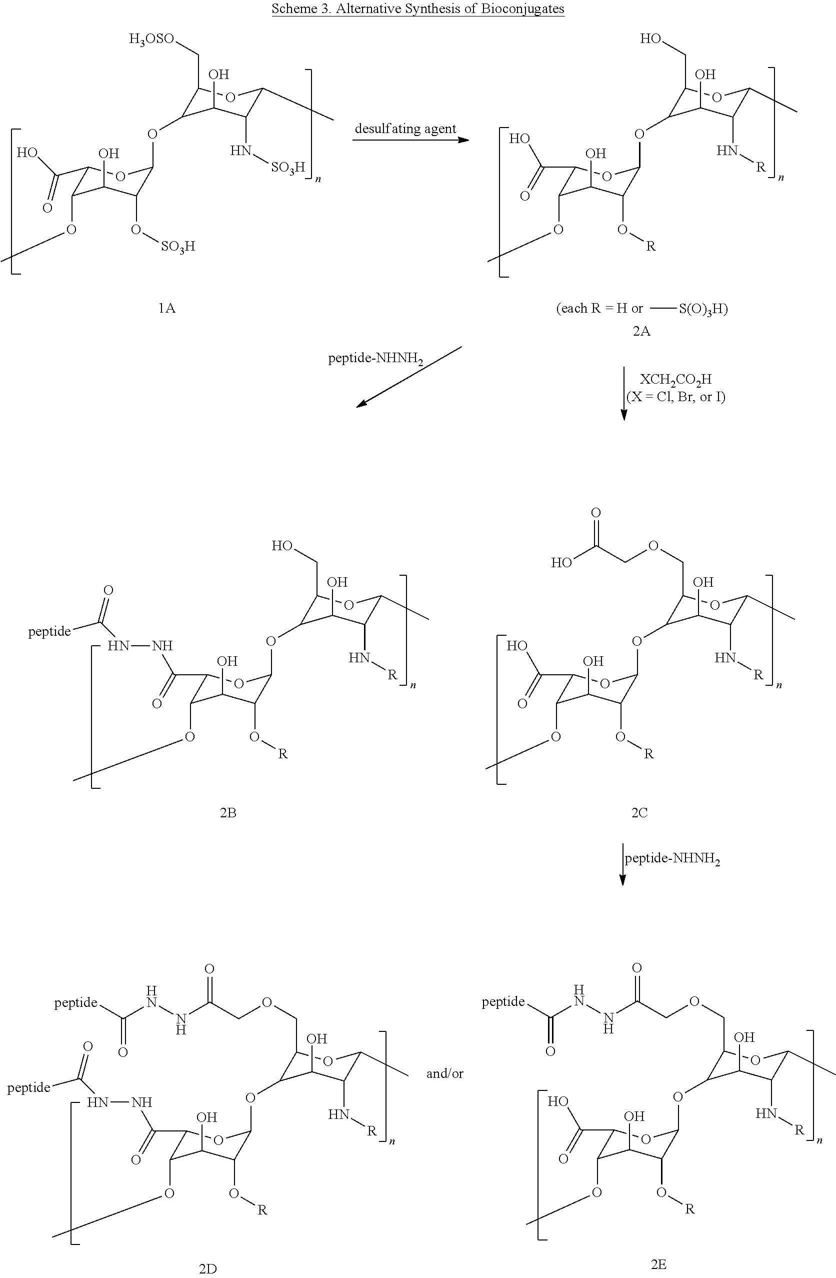

### 4. METHODS

Provided herein are exemplary disease categories (with specific diseases) where vascular permeability (plus microvascular injury and/or endothelial dysfunction) may be treated with the bioconjugate described herein alone, or in combination with another bioconjugate (e.g., a collagen-binding bioconjugate).

### A. Endothelial Dysfunction

The present disclosure, in one embodiment, provides bioconjugates, compositions and methods for treating a patient suffering from a disease associated with endothelial dysfunction. See, e.g., Lampugnani, M. G., Cold Spring Harbor perspectives in medicine 2012, 2(10), a006528, Dejana, E., Current opinion in hematology, 2012, 19(3), 218-223, Giannotta, M., Developmental cell, 2013, 26(5), 441-454, and Vestweber, D., Trends in cell biology, 2009, 19(1), 8-15.

Also provided, in some embodiments, is a method for preventing or reducing inflammation at a vascular site of a patient suffering from endothelial dysfunction. The method comprises administering to the site a pharmaceutical composition that includes a bioconjugate of the present disclosure.

The term “endothelial dysfunction” is also referred to as “endothelial cell (EC) dysfunction,” “dysfunctional endothelium,” or “dysfunctional endothelial cells,” and refers to the unmasking or exposure of ICAM and VCAM receptors, as well as, selectin receptors on the cell surface of an endothelial cell. P-selectin and E-selectin are examples of selectin receptors exposed which are transiently expressed on the cell surface due to damage and inflammation, and chronically expressed in dysfunctional endothelium. In certain embodiments, the endothelial dysfunction may be due to endothelial inflammation. An example of a disease state with chronic dysfunctional endothelial cells is diabetes.

In some embodiments, endothelial dysfunction is characterized with permeated endothelial lining or damaged endothelial cells. In some embodiments, the endothelial dysfunction is characterized by loss of glycocalyx. In some embodiments, the endothelial dysfunction is characterized by a selectin protein expressed on the surface of endothelial cells and exposed to circulation. In some embodiments, the site suffers from inflammation.

In one aspect, the vascular site is not denuded by physical means and is not undergoing or recovering from a vascular intervention procedure. Non-limiting examples of vascular intervention procedures include percutaneous coronary intervention (PCI). In certain embodiments, the vascular intervention procedure comprises denuding a blood vessel. In certain embodiments, the endothelial dysfunction is characterized by permeated endothelial lining or damaged endothelial cells. In certain embodiments, the endothelial dysfunction is characterized by loss of glycocalyx. In certain embodiments, the endothelial dysfunction is characterized by a selectin protein expressed on the surface of endothelial cells and exposed to circulation. In certain embodiments, the site suffers from inflammation. In certain embodiments, the bioconjugate is administered to achieve a plasma concentration of peptide ligand from 20 μM to 1000 μM proximate the dysfunctional endothelium. In certain embodiments, the bioconjugate is administered to achieve a plasma concentration of peptide ligand from 100 μM to 400 μM proximate the dysfunctional endothelium.

Dysfunction of the endothelium plays an important role in the pathogenesis of a broad spectrum of diseases as endothelial cells participate in the maintenance of functional capillaries.

For instance, the endothelium is directly involved in peripheral vascular disease, stroke, heart disease, diabetes, insulin resistance, chronic kidney failure, tumor growth, metastasis, venous thrombosis, and severe viral infectious diseases (Rajendran et al., Int. J. Biol. Sci., 9:1057-1069, 2013).

A “disease associated with endothelial dysfunction,” as used herein, refers to a human disease or condition that is at least in part caused by endothelial dysfunction or that induces endothelial dysfunction. Treating a disease associated with endothelial dysfunction, accordingly, refers to the treatment of the disease, recovering the dysfunctional endothelium, or preventing or ameliorating conditions or symptoms arising from dysfunctional endothelium, such as inflammation, intimal hyperplasia, and thrombosis.

It is contemplated that the bioconjugates can be effectively delivered to any organ of a human patient. Therefore, the bioconjugates can be used to treat endothelial dysfunction that occurs at any of the organs and associated with any of the following diseases or conditions.

Ischemic Reperfusion.

Ischemic reperfusion (IR) occurs following multiple pathological conditions and surgical procedures including native vein and artery grafts, stroke, severe sepsis, and organ transplantation. The earliest events result in generation of intracellular free radicals, a process linked to endothelial dysfunction. Upon restoration of blood flow platelets and neutrophils bind to the vascular wall resulting in thrombus formation, inflammation, neointimal thickening and general fibrosis. Endothelial selectins and cell adhesion molecules, ICAM and VCAM, are upregulated and the endothelial cells become inflamed, lose cell-cell contacts and expose underlying extracellular matrix.

Ischemia reperfusion injury is one of the leading causes of acute kidney injury. The vulnerability of the kidney is highlighted by the fact that it is one of the first organs to fail in septic patients and high failure rates in kidney transplantation. As a result of ischemic reperfusion endothelial dysfunction occurs, which is characterized in part by the loss of tight endothelial barrier function. Tight barrier function is lost when cell-cell contacts between endothelial cells fail. One of the key receptor molecules involved in tight junctions is VE-cadherin. When tight junctions are lost, not only due VE-cadherin molecules dissociate, but the protein begins to degrade making it challenging for the endothelial cells to reform the cell-cell contacts and the endothelial barrier. With loss of cell-cell contacts, extracellular matrix (ECM) is exposed and can serve as a site for thrombus formation.

Provided herein is a method for treating or preventing ischemic reperfusion injury in a patient in need thereof, comprising administering to the patient an effective amount of a bioconjugate or composition provided herein. In one embodiment, the ischemic reperfusion injury is a result of organ transplant (e.g., kidney, heart, liver, and vein graft). See, e.g., Reinders et al. Journal of the American Society of Nephrology, 2006, 17(4), 932-942. In one embodiment, the ischemic reperfusion injury is a result of arterial occlusion (e.g., peripheral, cardiac, neurologic). See, e.g., Callow, A. D., et al. Growth factors, 1994, 10(3), 223-228. In one embodiment, the ischemic reperfusion injury is a result of coronary bypass surgery. See, e.g., Li, J. et al. Journal of molecular and cellular cardiology, 2012, 52(4), 865-872. In one embodiment, the ischemic reperfusion injury is a result of a tourniquet and/or crush injury. See, e.g., Gillani, S., et al. Injury, 2012, 43(6), 670-675. In one embodiment, the ischemic reperfusion injury is a result of multi-organ failure (e.g., post CPR, sepsis syndrome, hemorrhage). In one embodiment, the ischemic reperfusion injury is a result of neonatal hypoxic-ischemic brain injury (periventricular leukomalacia, etc). See, e.g., Baburamani, A. A., et al.” Frontiers in physiology, 2012, 3, and Falahati, S., et al. Developmental neuroscience, 2013, 35(2-3), 182-196.

In any of the methods described herein, the organ or treatment site can be perfused with a bioconjugate or composition as provided herein prior to, at the time of, and/or periodically after reperfusion.

Vascular diseases. Vascular diseases that can be suitably treated with bioconjugates include, without limitation, atherosclerotic diseases (peripheral artery disease, coronary artery disease, stroke, carotid artery disease, renal arterial stenosis), venous thrombotic diseases (deep or superficial vein thrombosis), and iatrogenic large vessel injury (angioplasty, angioplasty with stent placement, atherectomy, thrombectomy, dialysis access creation, vein harvesting for bypass, treatment of brain or aortic aneurysms).

Renal diseases. Renal diseases that can be suitably treated with bioconjugates include, without limitation, acute tubular necrosis, diabetic chronic renal failure, lupus nephritis, renal fibrosis, and acute glomerulonephritis.

Pulmonary diseases. Pulmonary diseases that can be suitably treated with bioconjugate include, without limitation, idiopathic pulmonary fibrosis (IPF), chronic obstructive pulmonary disease, asthma, and emphysema. Also provided are methods for treating diseases or conditions that result in pulmonary distress, such as high-altitude pulmonary edema, pancreatitis, sepsis, or viral infections including, but not limited to, Ebola, Dengue fever, influenza, or Hantavirus.

Hematological diseases. Hematological diseases that can be suitably treated with bioconjugates include, without limitation, thrombotic thrombocytopenic purpura (TTP), disseminated intravascular coagulation (DIC), and hemolytic uremic syndrome (HUS).

Dermal Diseases. Dermal diseases that can be suitably treated with bioconjugates include, without limitation, systemic sclerosis.

Rheumatologic diseases. Rheumatologic diseases that can be suitably treated with bioconjugates include, but are not limited to, vasculitic disorders (lupus), rheumatoid arthritis and other inflammatory arthritis (gout).

Gastrointestinal Diseases. Gastrointestinal diseases that can be suitably treated with bioconjugates include, without limitation, inflammatory bowel disease, hepatitis, hepatic fibrosis, tumor growth, tumor metastasis, infectious diseases including viral and bacterial sepsis.

Neurologic Diseases. Neurologic diseases that can be suitably treated with bioconjugates include, without limitation, multiple sclerosis, dementia, and amyotrophic lateral sclerosis.

Ophthalmologic Diseases. Ophthalmologic diseases that can be suitably treated with bioconjugates include, without limitation, macular degeneration, glaucoma, and uveitis.

Endocrinological Diseases. Endocrinological diseases that can be suitably treated with bioconjugates include, without limitation, such as diabetes, and complex regional pain syndrome (CRPS).

It is contemplated that the bioconjugates provided herein, and compositions comprising the same, may also be capable of inhibiting inflammation due to dysfunctional endothelium.

### B. Fibrosis

Fibrosis is an inflammatory disease in which inflammatory cells migrate into tissue and organs, and leading to cellular responses that result in scarring. By preventing inflammatory cell extravasation, fibrosis can be attenuated or prevented.

Fibrosis can occur in many tissues within the body, typically as a result of inflammation or damage. In lungs, types of fibrosis include pulmonary fibrosis such as cystic fibrosis and idiopathic pulmonary fibrosis. Pulmonary fibrosis is a respiratory disease in which scars are formed in the lung tissues, leading to serious breathing problems. Scar formation leads to thickening of the walls, and causes reduced oxygen supply in the blood. As a consequence patients suffer from perpetual shortness of breath.

Cirrhosis is fibrosis in the liver in which the liver does not function properly due to long-term damage. Typically, the disease comes on slowly over months or years. Early on, there are often no symptoms. As the disease worsens, a person may become tired, weak, itchy, have swelling in the lower legs, develop yellow skin, bruise easily, have fluid buildup in the abdomen, or develop spider-like blood vessels on the skin. The fluid build-up in the abdomen may become spontaneously infected. Other complications include hepatic encephalopathy, bleeding from dilated veins in the esophagus or dilated stomach veins, and liver cancer. Hepatic encephalopathy results in confusion and possibly unconsciousness.

Cirrhosis can result in liver dysfunction. The following symptoms or features are direct consequences of liver dysfunction and thus can also be treated or ameliorated by the presently disclosed compositions and methods. Spider angiomata or spider nevi are vascular lesions consisting of a central arteriole surrounded by many smaller vessels and occur due to an increase in estradiol. Palmar erythema is a reddening of palms at the thenar and hypothenar eminences also as a result of increased estrogen. Gynecomastia, or increase in breast gland size in men that is not cancerous, is caused by increased estradiol and can occur in up to ⅔ of patients. Hypogonadism, a decrease in sex hormones manifest as impotence, infertility, loss of sexual drive, and testicular atrophy, can result from primary gonadal injury or suppression of hypothalamic/pituitary function. Hypogonadism is associated with cirrhosis due to alcoholism and hemochromatosis. Liver size can be enlarged, normal, or shrunken in people with cirrhosis.

Ascites, accumulation of fluid in the peritoneal cavity, gives rise to flank dullness. This can be visible as increase in abdominal girth. Fetor hepaticus is a musty breath odor resulting from increased dimethyl sulfide. Jaundice is yellow discoloration of the skin and mucous membranes due to increased bilirubin. In addition, liver cirrhosis increases resistance to blood flow and higher pressure in the portal venous system, resulting in portal hypertension.

In the heart, fibrosis is present in the form of atrial fibrosis, endomyocardial fibrosis, or myocardial infarction. Glial scar is fibrosis in the brain. Other types of fibrosis include, without limitation, arthrofibrosis (knee, shoulder, other joints), Crohn's disease (intestine), Dupuytren's contracture (hands, fingers), keloid (skin), mediastinal fibrosis (soft tissue of the mediastinum), myelofibrosis (bone marrow), Peyronie's disease (penis), nephrogenic systemic fibrosis (skin), progressive massive fibrosis (lungs), retroperitoneal fibrosis (soft tissue of the retroperitoneum), scleroderma/systemic sclerosis (skin, lungs), and some forms of adhesive capsulitis (shoulder).

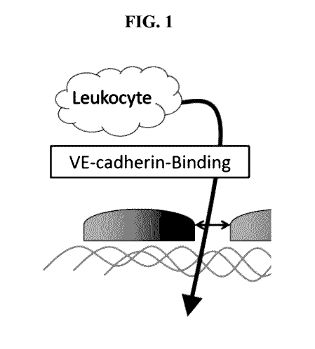

It is contemplated that the bioconjugates provided herein, and compositions comprising the same, may be effective in treating fibrosis by mitigating inflammation caused by endothelial cell-cell barrier loss, and subsequent leukocyte extravasation. In such embodiments, it is contemplated that the peptide conjugated to a glycan (such as heparin or dermatan sulfate), binds to VE-cadherin, which is a key glycoprotein responsible for maintaining endothelial cell-cell junctions. By binding to VE-cadherin, the bioconjugates prevent the loss of intercellular endothelial cell junctions, and by preserving cell junctions, inflammatory cells are inhibited from migrating into the subendothelial tissue by way of gaps between cells (see FIG. 1).

In another embodiment, the bioconjugates or compositions as provided herein having both VE-cadherin and collagen-binding properties, may mitigate leukocyte extravasation by recruitment of platelets bound on subendothelial collagen. In certain instances, it may be the case that endothelial cell junctions have already been compromised, and subsequently, subendothelial collagen is exposed. Platelets bind and activate on collagen, and subsequently recruit inflammatory cells, which migrate through the vessel and into the underlying tissue. In such embodiments, it is contemplated that the bioconjugates or compositions as provided herein having both VE-cadherin and collagen-binding properties would bind to subendothelial collagen, thus preventing platelet binding and activation, and ultimately preventing inflammatory cell extravasation into the tissue.

Accordingly, provided herein is a bioconjugate comprising at least one peptide comprising a VE-cadherin-binding unit and at least one peptide comprising a collagen-binding unit, in which are capable of both preserving endothelial cell-cell junctions, as well as preventing platelet-collagen interactions. Alternatively, a composition comprising two or more bioconjugates, where at least one comprises a VE-cadherin-binding unit and at least one comprises a collagen-binding unit, can be delivered to address each mechanism independently.

It is contemplated that the compositions and methods of the present disclosure are suitable for preventing and/or treating any of these diseases or symptoms or features associated with these diseases. Development of fibrosis involves stimulated cells laying down connective tissue, including collagen and glycosaminoglycans. The bioconjugates of the present disclosure can interact with the collagen or glycosaminoglycans and thus disrupt the formation of such excessive connective tissue. Accordingly, the bioconjugates can prevent, inhibit, delay, and/or reverse fibrosis.

In certain embodiments, the fibrosis is post ischemic, post infectious, or idiopathic (e.g., renal, hepatic, cardiac, pulmonary). See, e.g., Guerrot, D., et al. Fibrogenesis & tissue repair 5. Suppl 1 (2012): S15, and Yamaguchi, I., et al. Nephron Experimental Nephrology 120.1 (2012): e20-e31. In certain embodiments, the fibrosis is retroperitoneal. In certain embodiments, the fibrosis is dermal (e.g., scleroderma). See, e.g., Maurer, B., et al. Annals of the rheumatic diseases (2013): annrheumdis-2013.

### C. Other

### Neurovascular

It is contemplated that the bioconjugates and compositions of the present disclosure can be used in treating neurovascular disorders. Exemplary neurovascular disorders include, but are not limited to, chronic inflammatory demyelinating polyradiculoneuropathy (CIDP) (See, e.g., Van den Bergh, P. Y. K., et al. La Presse Médicale 42.6 (2013): e203-e215), MS (e.g., RRMS, PPMS) (See, e.g., Habets, K. L. L., et al. European journal of clinical investigation 43.7 (2013): 746-757), ALS (See, e.g., Winkler, E. A., et al. Acta neuropathologica 125.1 (2013): 111-120), HIV neurocognitive decline (See, e.g., Davidson, J Neuroinflammation 10.144 (2013): 11), stroke (ischemic), dementia (vascular type) (See, e.g., Nelson, A. R., et al. Biochimica et Biophysica Acta (BBA)-Molecular Basis of Disease (2015), concussion/CTE (See, e.g., Toklu, H. Z., et al. in Oxidative Stress, Brain Edema, Blood-Brain Barrier Permeability, and Autonomic Dysfunction from Traumatic Brain Injury (2015)), cavernous malformations (See, e.g., Dejana, E., et al. Developmental cell 16.2 (2009): 209-221), spinal cord injury (See, e.g., Oudega, M. Cell and tissue research 349.1 (2012): 269-288), encephalomyelitis (See, e.g., Imeri, F., et al. Neuropharmacology 85 (2014): 314-327), epilepsy, schizophrenia, mania (See, e.g., Levite, M. Journal of Neural Transmission 121.8 (2014): 1029-1075), cerebral edema (See, e.g., Schwarzmaier, S., et al. Journal of neurotrauma (2015)), meningitis (See, e.g., Erickson, M. A., et al., Neuroimmunomodulation 19.2 (2012): 121-130), moyamoya (See, e.g., Young, A. M. H., et al. Frontiers in neurology 4 (2013)), high-altitude cerebral edema, and hereditary haemorrhagic telangiectasia (See, e.g., Shovlin, C. L., et al. Thorax 54.8 (1999): 714-729).

### Vasculitis/Auto-Immune Diseases and Disorders

It is contemplated that the bioconjugates and compositions of the present disclosure can be used in treating vasculitis and/or auto-immune diseases and disorders. Exemplary diseases and disorders include, but are not limited to, lupus (e.g., renal, neuro, cutaneous, cardiac) (See, e.g., Habets, K. L. L., European journal of clinical investigation 43.7 (2013): 746-757), Churg-Strauss vasculitis, granulomatosis with polyangiitis (See, e.g., Hernandez, N. Transplantation (2015)), IgA vasculitis (Henoch-Schönlein purpura), Henoch-Schönlein purpura or Behcet's syndrome (See, e.g., Chen, T., et al. Rheumatology international 34.8 (2014): 1139-1143), scleroderma, such as skin, lung and renal crisis (See, e.g., Szucs, G., et al. Rheumatology 46.5 (2007): 759-762), and inflammatory bowel disease (See, e.g., Roifman, I., et al. Clinical Gastroenterology and Hepatology 7.2 (2009): 175-182).

### Ophthalmology

It is contemplated that the bioconjugates and compositions of the present disclosure can be used in treating ophthamologic diseases and disorders. Exemplary diseases and disorders include, but are not limited to, ocular auto-immune disease (e.g., uveitis) (See, e.g., Miller, J. W., et al. Ophthalmology 120.1 (2013): 106-114), macular degeneration (See, e.g., Kinnunen, K., et al. Acta ophthalmologica 90.4 (2012): 299-309), glaucoma (See, e.g., Coca-Prados, M. Journal of glaucoma 23 (2014): S36-S38), diabetic retinopathy (See, e.g., Yun, J-S., et al. Diabetes & metabolism journal 37.4 (2013): 262-269), and corneal transplant (See, e.g., Kuo, A. N., et al. American journal of ophthalmology 145.1 (2008): 91-96).

### Atherosclerosis

It is contemplated that the bioconjugates and compositions of the present disclosure can be used in treating atherosclerotic diseases and disorders. Exemplary diseases and disorders include, but are not limited to, post intervention for arterial occlusion (e.g., angioplasty, stent, atherectomy; PAD, coronary, carotid, aorta, renal, neurological, etc.) (See, e.g., Callow, A. D., et al. Growth factors 10.3 (1994): 223-228), critical limb ischemia (See, e.g., Dormandy, J. A., et al. Springer Science & Business Media, 2012), vein graft (e.g., PAD, CABG), AV fistula or graft placement or post intervention (See, e.g., Chiu, J-J, et al. Physiological reviews 91.1 (2011): 327-387), and diabetes (See, e.g., Widlansky, M. E., et al. Journal of the American College of Cardiology 42.7 (2003): 1149-1160).

### Renal

It is contemplated that the bioconjugates and compositions of the present disclosure can be used in treating renal diseases and disorders. Exemplary diseases and disorders include, but are not limited to, acute renal failure (e.g., ATN 0 acute tubular necrosis from contrast nephropathy) (see, e.g., Sutton, Timothy A. Microvascular research 77.1 (2009): 4-7), diabetic nephropathy (see, e.g., Bakker, Wineke, et al. Cell and tissue research 335.1 (2009): 165-189), and auto-immune nephropathy (See, e.g., Mayadas, T. N., et al. Circulation 120.20 (2009): 2012-2024).

Acetaminophen toxicity has replaced viral hepatitis as the most common cause of acute hepatic failure and is the second most common cause of liver failure requiring transplantation. It is also contemplated that the bioconjugates and compositions of the present disclosure can be used in treating acetaminophen hepatic toxicity/overdose.

### Systemic Syndromes

It is contemplated that the bioconjugates and compositions of the present disclosure can be used in treating systemic syndromes. Exemplary systemic syndromes include, but are not limited to, sepsis (any cause) (see, e.g., Madoiwa, Journal of Intensive Case, 2015, 3(8), 1-8), infection (sepsis) parainfluenza, adenoviruses, herpes simplex virus (HSV), polio virus, echovirus, measles virus, mumps virus, cytomegalovirus (CMV), human T-cell leukaemia virus type-1 (HTLV-1), human immunodeficiency virus (HIV), infections, such as Filovirus (e.g., dengue, dengue haemorrhagic shock, haemorrhagic shock, ebola, vascular leak syndrome (see, e.g., Wolf, et al., Lancet 2015, 385, 1428-1435 and Wahl-Jensen, et al., J Virol, 2005; 79(16): 10442-10450)), marburg, Hantaan and Lassa H F, leptospirosis, especially Weil's syndrome, Coxsackie B virus (see, e.g., Spiropoulou, C. F., et al. Virulence 4.6 (2013): 525-536 and Keller, Tymen T., et al. Cardiovascular research 60.1 (2003): 40-48), disseminated intravascular coagulation (DIC) (see, e.g., Wada, H., et al. Thrombosis research 125.1 (2010): 6-11), hemolytic uremic syndrome (HUS) (see, e.g., HUS, Shiga Toxin-Associated “The pathogenesis and treatment of hemolytic uremic syndrome.” (1998)), thrombotic thrombocytopenic purpura (TTP) (see, e.g., Tsai, H-M. Hematology/oncology clinics of North America 27.3 (2013): 565-584), pre-eclamsia (see, e.g., Powe, C. E., et al. Circulation 123.24 (2011): 2856-2869 and Uddin, M. N., et al. American journal of nephrology 30.1 (2009): 26-33), HELLP syndrome (hemolysis, elevated liver enzyme levels, and low platelet levels) (see, e.g., Jebbink, J., et al. Biochimica et Biophysica Acta (BBA)-Molecular Basis of Disease 1822.12 (2012): 1960-1969), Complex Regional Pain Syndrome (CRPS) (see, e.g., Ostergaard, L., et al. PAIN® 155.10 (2014): 1922-1926), ARDS (see, e.g., Mammoto, et al., Nature Comm, 2013, 4(1759) 1-10), hantavirus (see, e.g., Gavrilovskaya, J. Virol. 2008, 82(12), 5797-5806), bio weapons, such as anthrax (see, e.g., Liu, et al., J Cell Physiol. 2012; 227(4):1438-45), ricin (see, e.g., Lindstrom, et al., Blood, 1997, 90(6), 2323-2334) and DIC/TTP (see, e.g., Semeraro, et al., Endothelial Cell Perturbation and Disseminated Intravascular Coagulation, Landes Bioscience; 2000-2013) and systemic capillary leak syndrome (see, e.g., Xie, Z., et al. Blood 119.18 (2012): 4321-4332). It is contemplated that the bioconjugates and compositions of the present disclosure can be used in treating pancreatitis or influenza.

### Pulmonary

It is contemplated that the bioconjugates and compositions of the present disclosure can be used in treating pulmonary diseases and disorders. Exemplary pulmonary diseases and disorders include, but are not limited to, ARDS (see, e.g., Phillips, C. R., et al. Critical care medicine 36.1 (2008): 69-73, Maniatis, N. A., et al. Current opinion in critical care 14.1 (2008): 22-30, and Aman, J., et al. Critical care medicine 39.1 (2011): 89-97), COPD (see, e.g., Olivieri, D., et al. “Therapeutic perspectives in vascular remodeling in asthma and chronic obstructive pulmonary disease.” (2014): 216-225 and Moro, L., et al. Angiology (2008)), CF (see, e.g., Poore, S., et al. CHEST Journal 143.4 (2013): 939-945), Primary Pulm HTN (see, e.g., Budhiraja, R. et al. Circulation 109.2 (2004): 159-165), allergic pneumonitis, pulmonary A-V malformations (see, e.g., Shovlin, C. L., et al. Thorax 54.8 (1999): 714-729), and asthma (see, e.g., Olivieri, D. “Therapeutic perspectives in vascular remodeling in asthma and chronic obstructive pulmonary disease.” (2014): 216-225).

### Trauma

It is contemplated that the bioconjugates and compositions of the present disclosure can be used in treating trauma or traumatic injuries. Exemplary traumatic injuries include, but are not limited to, concussion/CTE (see, e.g., Shetty, et al., Front Cell Neurosci. 2014; 8: 232), crush injury, ischemia reperfusion, or rhabdomyolysis-kidney injury (see, e.g., Blaisdell, Vascular, 2002, 10(6), 620-630), spinal cord injury (see, e.g., Figley, et al. J Neurotrauma 2014; 31(6): 541-552), complex regional pain syndrome (CRPS) (see, e.g., see, e.g., Ostergaard, L., et al. PAIN, 155.10 (2014): 1922-1926), corneal injury (see, e.g., Ashby, Austin J Clin Ophthalmol 2014; 1(4): 1017), or others, such as, burns, cerebral edema, etc.

### Combination Therapy

In some embodiments, the bioconjugates and compositions of the present disclosure can be used in combination with a second agent useful for preventing or treating fibrosis. Accordingly, in one embodiment, a combination, pharmaceutical composition, package or kit is provided that includes any pharmaceutical composition of the present disclosure and one or more such second agent. In one embodiment, any treatment method of the present disclosure further includes administration of one or more such second agent.

The second agent can be any pharmaceutical or biologic agent that is useful for preventing, treating or otherwise ameliorating symptoms of fibrosis. Non-limiting examples include steroids such as predonine, reducing agents such as N-acetylcysteine, antifibrotic drugs such as pirfenidone and nintedanib, immunosuppressive drugs such as corticosteroids, cyclophosphamide, azathioprine, methotrexate, penicillamine, and cyclosporine A and FK506, and other agents like colchicine, IFN-γ and mycophenolate mofetil.

### 5. PHARMACEUTICAL COMPOSITIONS

In one embodiment, the bioconjugate is administered in a pharmaceutical composition. The present disclosure provides pharmaceutical compositions comprising a bioconjugate, or a composition comprising the same, and a pharmaceutically acceptable carrier. Pharmaceutically acceptable carriers are known to one having ordinary skill in the art may be used, including water or saline. As is known in the art, the components as well as their relative amounts are determined by the intended use and method of delivery. The pharmaceutical compositions provided in accordance with the present disclosure are formulated as a solution for delivery into a patient in need thereof. Diluent or carriers employed in the pharmaceutical compositions can be selected so that they do not diminish the desired effects of the bioconjugate. Examples of suitable pharmaceutical compositions include aqueous solutions, for example, a solution in isotonic saline, 5% glucose. Other well-known pharmaceutically acceptable liquid carriers such as alcohols, glycols, esters and amides, may be employed. In certain embodiments, the pharmaceutical composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents.

In certain embodiments, a polymer matrix or polymeric material is employed as a pharmaceutically acceptable carrier or support for the pharmaceutical composition. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, the pharmaceutical compositions provided herein is formulated as films, gels, foams, or and other dosage forms.

Suitable ionic strength modifying agents include, for example, glycerin, propylene glycol, mannitol, glucose, dextrose, sorbitol, sodium chloride, potassium chloride, and other electrolytes.

In certain embodiments, the solubility of the bioconjugate may need to be enhanced. In such cases, the solubility may be increased by the use of appropriate formulation techniques, such as the incorporation of solubility-enhancing pharmaceutical compositions such as mannitol, ethanol, glycerin, polyethylene glycols, propylene glycol, poloxomers, and others known in the art.

In certain embodiments, the pharmaceutical composition contains a lubricity enhancing agent. As used herein, lubricity enhancing agents refer to one or more pharmaceutically acceptable polymeric materials capable of modifying the viscosity of the pharmaceutically acceptable carrier. Suitable polymeric materials include, but are not limited to: ionic and non-ionic water soluble polymers; hyaluronic acid and its salts, chondroitin sulfate and its salts, dextrans, gelatin, chitosans, gellans, other bioconjugate or polysaccharides, or any combination thereof; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); gums such as tragacanth and xanthan gum; gellan gums; alginate and sodium alginate; chitosans; vinyl polymers; hydrophilic polymers such as polyethylene oxides, polyoxyethylene-polyoxypropylene copolymers, and polyvinylalcohol; carboxyvinyl polymers or crosslinked acrylic acid polymers such as the “carbomer” family of polymers, e.g., carboxypolyalkylenes that may be obtained commercially under the Carbopol™ trademark; and various other viscous or viscoelastomeric substances. In one embodiment, a lubricity enhancing agent is selected from the group consisting of hyaluronic acid, dermatan, chondroitin, heparin, heparan, keratin, dextran, chitosan, alginate, agarose, gelatin, hydroxypropyl cellulose, hydroxyethyl cellulose, hydroxypropyl methylcellulose, hydroxypropyl methylcellulose phthalate, methyl cellulose, carboxymethyl cellulose, and etherified cellulose, polyvinyl alcohol, polyvinylpyrrolidinone, povidone, carbomer 941, carbomer 940, carbomer 971P, carbomer 974P, or a pharmaceutically acceptable salt thereof. In one embodiment, a lubricity enhancing agent is applied concurrently with the bioconjugate. Alternatively, in one embodiment, a lubricity enhancing agent is applied sequentially to the bioconjugate. In one embodiment, the lubricity enhancing agent is chondroitin sulfate. In one embodiment, the lubricity enhancing agent is hyaluronic acid. The lubricity enhancing agent can change the viscosity of the pharmaceutical composition.

For further details pertaining to the structures, chemical properties and physical properties of the above lubricity enhancing agents, see e.g., U.S. Pat. No. 5,409,904, U.S. Pat. No. 4,861,760 (gellan gums), U.S. Pat. No. 4,255,415, U.S. Pat. No. 4,271,143 (carboxyvinyl polymers), WO 94/10976 (polyvinyl alcohol), WO 99/51273 (xanthan gum), and WO 99/06023 (galactomannans). Typically, non-acidic lubricity enhancing agents, such as a neutral or basic agent are employed in order to facilitate achieving the desired pH of the pharmaceutical composition.

In some embodiments, the bioconjugates can be combined with minerals, amino acids, sugars, peptides, proteins, vitamins (such as ascorbic acid), or laminin, collagen, fibronectin, hyaluronic acid, fibrin, elastin, or aggrecan, or growth factors such as epidermal growth factor, platelet-derived growth factor, transforming growth factor beta, or fibroblast growth factor, and glucocorticoids such as dexamethasone or viscoelastic altering agents, such as ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic.

Suitable pH buffering agents for use in the pharmaceutical compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or IVIES. In certain embodiments, an appropriate buffer system (e.g., sodium phosphate, sodium acetate, sodium citrate, sodium borate or boric acid) is added to the pharmaceutical composition to prevent pH drift under storage conditions. In some embodiments, the buffer is a phosphate buffered saline (PBS) solution (i.e., containing sodium phosphate, sodium chloride and in some formulations, potassium chloride and potassium phosphate). The particular concentration will vary, depending on the agent employed. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8. In some embodiments, the buffer is chosen to maintain a pH within the range of from about pH 4 to about pH 8. In some embodiments, the pH is from about pH 5 to about pH 8. In some embodiments, the buffer is a saline buffer. In certain embodiments, the pH is from about pH 4 and about pH 8, or from about pH 3 to about pH 8, or from about pH 4 to about pH 7. In some embodiments, the pharmaceutical composition is in the form of a film, gel, patch, or liquid solution which comprises a polymeric matrix, pH buffering agent, a lubricity enhancing agent and a bioconjugate wherein the pharmaceutical composition optionally contains a preservative; and wherein the pH of said pharmaceutical composition is within the range of about pH 4 to about pH 8.

Surfactants are employed in the pharmaceutical composition to deliver higher concentrations of bioconjugate. The surfactants function to solubilize the inhibitor and stabilize colloid dispersion, such as micellar solution, microemulsion, emulsion and suspension. Suitable surfactants comprise c polysorbate, poloxamer, polyoxyl 40 stearate, polyoxyl castor oil, tyloxapol, triton, and sorbitan monolaurate. In one embodiment, the surfactants have hydrophile/lipophile/balance (HLB) in the range of 12.4 to 13.2 and are acceptable for ophthalmic use, such as TritonX114 and tyloxapol.

In certain embodiments, stabilizing polymers, i.e., demulcents, are added to the pharmaceutical composition. The stabilizing polymer should be an ionic/charged example, more specifically a polymer that carries negative charge on its surface that can exhibit a zeta-potential of (−)10-50 mV for physical stability and capable of making a dispersion in water (i.e. water soluble). In one embodiment, the stabilizing polymer comprises a polyelectrolyte or polyectrolytes if more than one, from the family of cross-linked polyacrylates, such as carbomers and Pemulen®, specifically Carbomer 974p (polyacrylic acid), at a range of about 0.1% to about 0.5% w/w.

In one embodiment, the pharmaceutical composition comprises an agent which increases the permeability of the bioconjugate to the extracellular matrix of blood vessels. Preferably the agent which increases the permeability is selected from benzalkonium chloride, saponins, fatty acids, polyoxyethylene fatty ethers, alkyl esters of fatty acids, pyrrolidones, polyvinylpyrrolidone, pyruvic acids, pyroglutamic acids or mixtures thereof.

The bioconjugate may be sterilized to remove unwanted contaminants including, but not limited to, endotoxins and infectious agents. Sterilization techniques which do not adversely affect the structure and biotropic properties of the bioconjugate can be used. In certain embodiments, the bioconjugate can be disinfected and/or sterilized using conventional sterilization techniques including propylene oxide or ethylene oxide treatment, sterile filtration, gas plasma sterilization, gamma radiation, electron beam, and/or sterilization with a peracid, such as peracetic acid. In one embodiment, the bioconjugate can be subjected to one or more sterilization processes. Alternatively, the bioconjugate may be wrapped in any type of container including a plastic wrap or a foil wrap, and may be further sterilized.

In some embodiments, preservatives are added to the pharmaceutical composition to prevent microbial contamination during use. Suitable preservatives added to the pharmaceutical compositions comprise benzalkonium chloride, benzoic acid, alkyl parabens, alkyl benzoates, chlorobutanol, chlorocresol, cetyl alcohols, fatty alcohols such as hexadecyl alcohol, organometallic compounds of mercury such as acetate, phenylmercury nitrate or borate, diazolidinyl urea, diisopropyl adipate, dimethyl polysiloxane, salts of EDTA, vitamin E and its mixtures. In certain embodiments, the preservative is selected from benzalkonium chloride, chlorobutanol, benzododecinium bromide, methyl paraben, propyl paraben, phenylethyl alcohol, edentate disodium, sorbic acid, or polyquarternium-1. In certain embodiments, the pharmaceutical compositions contain a preservative. In some embodiments, the preservatives are employed at a level of from about 0.001% to about 1.0% w/v. In certain embodiments, the pharmaceutical compositions do not contain a preservative and are referred to as “unpreserved”. In some embodiments, the pharmaceutical compositions are sterile, but unpreserved.

Exemplary pharmaceutical compositions for use with the bioconjugates for catheter-based delivery may comprise: a) a bioconjugate as described herein; b) a pharmaceutically acceptable carrier; c) a polymer matrix; d) a pH buffering agent to provide a pH in the range of about pH 4 to about pH 8; and e) a water soluble lubricity enhancing agent in the concentration range of about 0.25% to about 10% total formula weight or any individual component a), b), c), d) ore), or any combinations of a), b), c), d) or e).

Pharmaceutical compositions contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In making pharmaceutical compositions that include bioconjugates described herein, the active ingredient is usually diluted by an excipient or carrier and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the pharmaceutical compositions can be in the form of films, gels, patches, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin films, gels, patches, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The pharmaceutical compositions can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

Films used for drug delivery are well known in the art and comprise non-toxic, non-irritant polymers devoid of leachable impurities, such as polysaccharides (e.g., cellulose, maltodextrin, etc.). In some embodiments, the polymers are hydrophilic. In certain embodiments, the polymers are hydrophobic. The film adheres to tissues to which it is applied, and is slowly absorbed into the body over a period of about a week. Polymers used in the thin-film dosage forms described herein are absorbable and exhibit sufficient peel, shear and tensile strengths as is well known in the art. In some embodiments, the film is injectable. In certain embodiments, the film is administered to the patient prior to, during or after surgical intervention.

Gels are used herein refer to a solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 90% water. In some embodiments, the gel described herein comprises a natural or synthetic polymeric network. In some embodiments, the gel comprises a hydrophilic polymer matrix. In certain embodiments, the gel comprises a hydrophobic polymer matrix. In some embodiments, the gel possesses a degree of flexibility very similar to natural tissue. In certain embodiments, the gel is biocompatible and absorbable. In certain embodiments, the gel is administered to the patient prior to, during or after surgical intervention.

Liquid solution as used herein refers to solutions, suspensions, emulsions, drops, ointments, liquid wash, sprays, liposomes which are well known in the art. In some embodiments, the liquid solution contains an aqueous pH buffer agent which resists changes in pH when small quantities of acid or base are added. In certain embodiments, the liquid solution is administered to the patient prior to, during or after surgical intervention.

Exemplary pharmaceutical compositions may comprise: a) bioconjugate as described herein; b) pharmaceutically acceptable carrier; c) polymer matrix; and d) pH buffering agent to provide a pH in the range of about pH 4 to about pH 8, wherein said solution has a viscosity of from about 3 to about 30 cps for a liquid solution. In certain embodiments, the solutions have a viscosity of from about 1 to about 100 centipoises (cps), or from about 1 to about 200 cps, or from about 1 to about 300 cps, or from about 1 to about 400 cps. In some embodiments, the solutions have a viscosity of from about 1 to about 100 cps. In certain embodiments, the solutions have a viscosity of from about 1 to about 200 cps. In certain embodiments, the solutions have a viscosity of from about 1 to about 300 cps. In certain embodiments, the solutions have a viscosity of from about 1 to about 400 cps.

Alternatively, exemplary pharmaceutical compositions may comprise: a) bioconjugate as described herein; b) pharmaceutically acceptable carrier; and c) hydrophilic polymer as matrix network, wherein said pharmaceuticalcompositions are formulated as viscous liquids, i.e., viscosities from several hundred to several thousand cps, gels or ointments. In these embodiments, the bioconjugate is dispersed or dissolved in an appropriate pharmaceutically acceptable carrier.

In certain embodiments, the bioconjugate, or a composition comprising the same, is lyophilized prior to, during, or after, formulation. In certain embodiments, the bioconjugate, or a composition comprising the same, is lyophilized in a pharmaceutical composition comprising a bulking agent, a lyoprotectant, or a mixture thereof. In certain embodiments, the lyoprotectant is sucrose. In certain embodiments, the bulking agent is mannitol. In certain embodiments, the bioconjugate, or a composition comprising the same, is lyophilized in a pharmaceutical composition comprising mannitol and sucrose. Exemplary pharmaceutical compositions may comprise about 1-20% mannitol and about 1-20% sucrose. The pharmaceutical compositions may further comprise one or more buffers, including but not limited to, phosphate buffers. Accordingly, also provided herein is a lyophilized composition comprising a bioconjugate or composition comprising the same as described herein.

### 6. DOSING & ADMINISTRATION

In various embodiments, the bioconjugates can be administered via any suitable route, e.g., intravenously, for delivery into the patient. Suitable routes for parenteral administration include intravascular, intravenous, intraperitoneal, intraarterial, intramuscular, cutaneous, subcutaneous, percutaneous, intradermal, and intraepidermal delivery. Suitable means for parenteral administration include needle (including microneedle) injectors, infusion techniques, and catheter-based delivery.

Pharmaceutical compositions of any of the bioconjugates described herein can be formulated for parenteral administration or catheter-based delivery. For example, such parenteral compositions can include:

a) a pharmaceutically active amount of one or more of the bioconjugates;

b) a pharmaceutically acceptable pH buffering agent to provide a pH in the range of about pH 4.5 to about pH 9;

c) an ionic strength modifying agent in the concentration range of about 0 to about 300 millimolar; and

d) water soluble viscosity modifying agent in the concentration range of about 0.25% to about 10% total formula weight or any individual component a), b), c), or d) or any combinations of a), b), c) and d) are provided.

In various embodiments described herein, the ionic strength modifying agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or basic agent are employed in order to facilitate achieving the desired pH of the parenteral composition.

In various embodiments described herein, parenteral compositions may be suitably formulated as a sterile non-aqueous solution or as a dried form to be used in conjunction with a suitable vehicle such as sterile, pyrogen-free water. The preparation of parenteral compositions under sterile conditions, for example, by lyophilization, may readily be accomplished using standard pharmaceutical techniques available to those skilled in the art.

In various embodiments described herein, the solubility of bioconjugates used in the preparation of a parenteral composition may be increased by the use of appropriate formulation techniques, such as the incorporation of solubility-enhancing compositions such as mannitol, ethanol, glycerin, polyethylene glycols, propylene glycol, poloxomers, and others known to those of skill in the art.

In various embodiments described herein, pharmaceutical compositions for parenteral administration may be formulated to be for immediate and/or modified release. Modified release compositions include delayed, sustained, pulsed, controlled, targeted and programmed release compositions. Thus, one or more bioconjugates may be formulated as a solid, semi-solid, or thixotropic liquid for administration as an implanted depot providing modified release of the active compound. Illustrative examples include drug-coated stents and copolymeric(dl-lactic, glycolic)acid (PGLA) microspheres. In another embodiment, one or more bioconjugates, or compositions comprising one or more bioconjugates, can be continuously administered, where appropriate, by IV drip, for example.

In any of the embodiments described herein, the bioconjugates can be delivered to the treatment site via a catheter (e.g., a dilatation catheter, an over-the-wire angioplasty balloon catheter, an infusion catheter, a rapid exchange or monorail catheter, or any other catheter device known in the art) which is percutaneously inserted into the patient and which is threaded through the patient's blood vessels to the target vessel. Various catheter-based devices are available in the art, including those described in U.S. Pat. No. 7,300,454, incorporated herein by reference. In another embodiment, the bioconjugates can be injected directly into the treatment site. In another embodiment, the bioconjugates can be delivered systemically (i.e., not delivered directly to the treatment site, but delivered by parenteral administration without catheter-based delivery). Illustratively, the catheter tip can be maintained stationary while bioconjugates are being delivered, or the catheter tip can be moved while the bioconjugates are being delivered (e.g., in a proximal direction from a position that is initially distal to the blockage, to or through the blockage, or to a position which is proximal to the blockage).

In any of the embodiments described herein, delivery of the bioconjugates can be continuous or it can be effected through a single or multiple administrations. Prior to, during, and/or after the bioconjugates are administered to the target site, the same bioconjugates or one or more different bioconjugates can be administered.

In any of the embodiments described herein, the bioconjugates, or composition thereof, can be administered alone or in combination with suitable pharmaceutical carriers or diluents. Diluent or carrier ingredients can be selected so that they do not diminish the desired effects of the bioconjugates. The bioconjugates, or composition thereof, may be in any suitable form. Examples of suitable dosage forms include aqueous solutions of the bioconjugates, for example, a solution in isotonic saline, 5% glucose or other well-known pharmaceutically acceptable liquid carriers such as alcohols, glycols, esters and amides.

The dosage of the bioconjugates can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition.

Any effective regimen for administering the bioconjugates can be used. For example, the bioconjugates can be administered as a single dose, or as a multiple-dose daily regimen. Further, a staggered regimen, for example, one to five days per week can be used as an alternative to daily treatment.

In various embodiments described herein, the patient is treated with multiple injections of the bioconjugates. In one embodiment, the patient is injected multiple times (e.g., about 2 up to about 50 times) with the bioconjugates, for example, at 12-72 hour intervals or at 48-72 hour intervals. Additional injections of the bioconjugates can be administered to the patient at an interval of days or months after the initial injections(s).

In some embodiments, the pharmaceutical compositions are formulated and packaged as an IV drip composition.

Suitable dosages of the bioconjugate can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.0001 mg to about 10 mg. In other illustrative aspects, effective doses ranges from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In any of the various embodiments described herein, effective doses ranges from about 0.01 μg to about 1000 mg per dose, 1 μg to about 100 mg per dose, about 100 μg to about 1.0 mg, about 50 μg to about 600 μg, about 50 μg to about 700 μg, about 100 μg to about 200 μg, about 100 μg to about 600 μg, about 100 μg to about 500 μg, about 200 μg to about 600 μg, or from about 100 μg to about 50 mg per dose, or from about 500 μg to about 10 mg per dose or from about 1 mg to about 10 mg per dose. In other illustrative embodiments, effective doses can be about 1 μg, about 10 μg, about 25 μg, about 50 μg, about 75 μg, about 100 μg, about 125 μg, about 150 μg, about 200 μg, about 250 μg, about 275 μg, about 300 μg, about 350 μg, about 400 μg, about 450 μg, about 500 μg, about 550 μg, about 575 μg, about 600 μg, about 625 μg, about 650 μg, about 675 μg, about 700 μg, about 800 μg, about 900 μg, 1.0 mg, about 1.5 mg, about 2.0 mg, about 10 mg, about 100 mg, or about 100 mg to about 30 grams. In certain embodiments, the dose is from about 0.01 mL to about 10 mL. In certain embodiments, the bioconjugate is administered via IV drip. In certain embodiments, the dose is from about 10 mL to about 1 L, or from about 10 mL to about 1 L, or from about 100 mL to about 1 L, or from about 200 mL to about 1 L, or from about 300 mL to about 1 L, or from about 400 mL to about 1 L, or from about 500 mL to about 1 L, or from about 600 mL to about 1 L, or from about 700 mL to about 1 L, or from about 800 mL to about 1 L, or from about 900 mL to about 1 L, or about 1 L.

In some embodiments, the pharmaceutical compositions are packaged in multidose form. Preservatives are thus required to prevent microbial contamination during use. In certain embodiments, suitable preservatives as described above can be added to the pharmaceutical compositions. In some embodiments, the pharmaceutical composition contains a preservative. In certain embodiments the preservatives are employed at a level of from about 0.001% to about 1.0% w/v. In some embodiments, the pharmaceutical compositions are sterile, but unpreserved.

In some embodiments, separate or sequential administration of the bioconjugate, or composition thereof, and another agent is necessary to facilitate delivery into the patient. In certain embodiments, the bioconjugate, or composition thereof, and another agent can be administered at different dosing frequencies or intervals. For example, the bioconjugate, or composition thereof, can be administered daily, while the other agent can be administered less frequently. Additionally, as will be apparent to those skilled in the art, the bioconjugate, or composition thereof, and another agent can be administered using the same route of administration or different routes of administration. In one embodiment, an effective amount of a pharmaceutical composition comprising a bioconjugate, or composition thereof, and pharmaceutically acceptable carrier is administered to a patient in need thereof, e.g., to treat a fibrotic disease or vascular disease or disorder, for instance, without limitation.

### Examples

### Example 1. Synthesis of Bioconjugates

Heparin (MW_{avg}=16 kDa) (purchased from Bioiberica, Spain) (20 mg/mL), Bbeta peptide (GHRPLDKKREEAPSLRPAPPPISGGGYR-hydrazide, 3 mg/mL) or (GHRPLDKKREEAPSLRPAPPPISGGGYRGSG-hydrazide, 3 mg/mL) (purchased from InnoPep, California), and EDC (75 mg/mL) were solubilized in an appropriate concentration of a chaotropic agent, such as butanol, ethanol, guanidinium chloride, lithium perchlorate, lithium acetate, magnesium chloride, phenol, propanol, sodium dodecyl sulfate, thiourea, or urea (e.g., from about 5 M to about 10 M urea), 0.064 M MES, 0.6% NaCl, pH 5.5. EDC was added to heparin at a molar ratio of 50:1 (EDC:heparin) and reacted for 5 minutes. Bbeta peptide was then added to activated heparin at a molar ratio of 8:1 (peptide:heparin) and reacted for 2 hours. The reaction was quenched by raising the pH to 8 using 0.5 M NaOH and holding for 30 minutes. eHep-Bbeta was then purified from urea and MES through weak anion exchange. The reaction was applied to a DEAE HiTrap FF column (GE Healthcare Life Sciences 17-5055-01), and the conjugate was eluted using a gradient of 0 to 2 M NaCl in 20 mM Tris, pH 8. The conjugate was then desalted through TFF with 12 CVs of water.

The measurement of ultraviolet absorbance by intrinsic chromophores is commonly used to predict peptide concentration. This method is particularly useful when absorbance is measured at 280 nm (A₂₈₀), and offers high specificity as the absorbance arises strictly from tryptophan and tyrosine residues. The peptide concentration is then easily determined using Beer's law: Absorbance=εLc where ε is molar extinction coefficient, L path length of the cell holder and c concentration of the solution.

The molar extinction coefficient of tyrosine and tryptophan at 280 nm were determined to be 1189 AU/mmole/ml and 5264 AU/mmole/ml respectively. A lyophilized sample of eHep-Bbeta was dissolved at 4 mg/ml and it's absorbance measured at 280 nm using a cuvette on a Nanodrop. The concentration was determined using the formula below:

$$Peptide concentration mg \text{/} ml = ( {A}_{280} \times MW ) \text{/} ɛ$$

$${A}_{280} is absorbance at 280 nm$$

$$MW is peptide molecular weight peptide mg \text{/} ml = \frac{0.62 AU * 3254.65 mg \text{/} mmole}{1189 AU \text{/} mmole \text{/} ml} = 1.697$$

The GAG concentration was the then determined by subtracting the peptide concentration from the eHep-Bbeta concentration. The peptide to GAG ratio was determined using the formula below:

$$Peptide \text{:} GAG = peptide molar concentration \text{/} GAG molar concentration$$

$${MW}_{Bbeta} = 3254.65 , {MW}_{Heparin} = 16200$$

$$Bbeta \text{:} Heparin = ( 1.697 \text{/} 3254.65 ) \text{/} ( 2.303 \text{/} 16200 ) = 3.668$$

Accordingly to the data, the eHep-Bbeta bioconjugate comprises about 3.7 peptides/heparin.

eDS-Bbeta was synthesized from dermatan sulfate (DS) (purchased from Bioiberica, Spain) (MW_{avg}=42 kDa) and Bbeta peptide (purchased from InnoPep, California) as described above using Bbeta peptide and activated DS at a molar ratio of 10:1 (peptide:DS).

### Example 2. VE-Cadherin Binding Assay

Recombinant VE cadherin/Fc chimera (R&D systems, Prod#938-VC) was prepared in 1× Phosphate Buffered Saline (PBS, Gibco, pH 7.4) at 5 μg/mL. 50 μL of this solution was incubated in each well of Costar high bind plate (Prod #9018) overnight at 4° C. The plate was washed three times with PBS. VE-cadherin coated wells were blocked using 0.5% non-fat dry milk and 50 μg/mL heparin sodium in 1×PBS. Blocked plates were then treated with a concentration gradient of eHep-Bbeta (biotinylated) molecule (eHep-Bbeta, 1:8:50). The eHep-Bbeta was dissolved in Tris-NaCl—CaCl₂ at 1 mg/mL, serial dilution (1:3) 50 μL 2 hour RT. At the end of the incubation plate was washed three times with PBS.

Ultra-Streptavidin HRP (ThermoFisher Scientific, Prod# N504) was diluted 1:500 using 1% BSA, 1×PBS and 0.05% tween was used to detect the biotinylated molecule bound to rh VE-cadherin coated plates. 100 μL of streptavidin solution was incubated in each well for 20 minutes at RT. The plate was washed three times with 1×PBS. 100 μL of TMB substrate solution (Abcam, slowest kinetic rate) was added to each well and developed for 20 minutes in the dark. 25 μL of stop solution (0.64 M H₂SO₄ in water) was added to stop the reaction and absorbance was read using Molecular Device i3 at 450 nm.

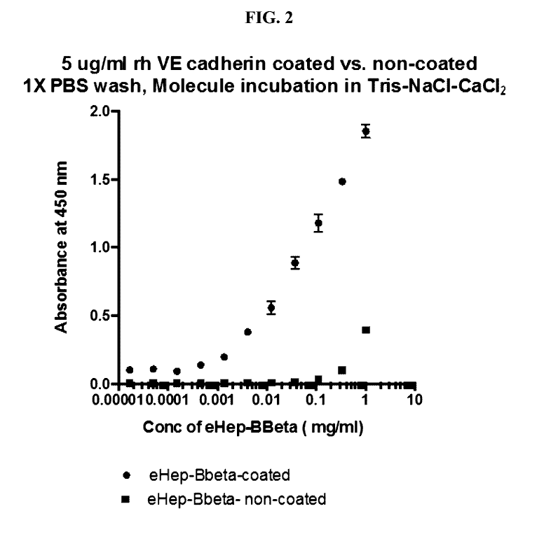

FIG. 2 shows that the bioconjugate described herein binds to VE-cadherin in a dose dependent manner. In addition, it was observed that a Tris-NaCl—CaCl₂ buffer enhances molecule binding significantly (see, e.g., Gorlatov, S., Biochemistry, 2002, 41(12), 4107-4116). This assay may be used to determine the binding affinity of the bioconjugates as well as the peptides alone.

### Example 3. HUVEC Culture

HUVECs were grown to confluence in 24-well tissue culture treated plates (10000 cells per cm²) for 3 days. The cells were cultured in vascular basal medium (ATCC, prod# PCS-100-030) supplemented with 0.2% bovine brain extract (BBE), 10 mM L-glutamine, 5 U/mL heparin sodium, 1 μg/mL hydrocortisone hemisuccinate, 50 μg/mL ascorbic acid, and 20% fetal bovine serum. The wells were washed three times with 1× phosphate buffered saline (PBS), and then were treated with medium alone, medium supplemented with thrombin at 1 U/mL, medium supplemented with thrombin at 1 U/mL and eHep-Bbeta at 100 μg/mL, medium supplemented with thrombin at 1 U/mL and heparin at 100 μg/mL for 10 minutes at 37° C.

The wells were then washed three times with 1×PBS. Each wash was for 5 minutes. The cells were then fixed using 4% formalin in 1×PBS for 15 minutes at room temperature, washed four times with 1×PBS (each wash was for 10 minutes). Blocking buffer containing 5% normal goat serum, and 0.3% Triton-X 100 in 1×PBS was prepared and 500 μL was added to each well. Blocking was done for 1 hour at room temperature.

Antibody dilution buffer containing 1% BSA, and 0.3% Triton-X 100 in 1×PBS was prepared. 200 μL of Rabbit anti pMLC diluted 1:50 using antibody dilution buffer was added to each well and incubated overnight at 4° C. The plate was washed 4 times with 1×PBS. Each wash was for 10 minutes.

Secondary antibody cocktail containing goat anti-rabbit cy5 (diluted 1:200) and alexa fluor 488 phalloidin (diluted 1:100) in antibody dilution buffer was prepared. 200 μL of this solution was added to each well and incubated for 2 hours at room temperature in the dark.

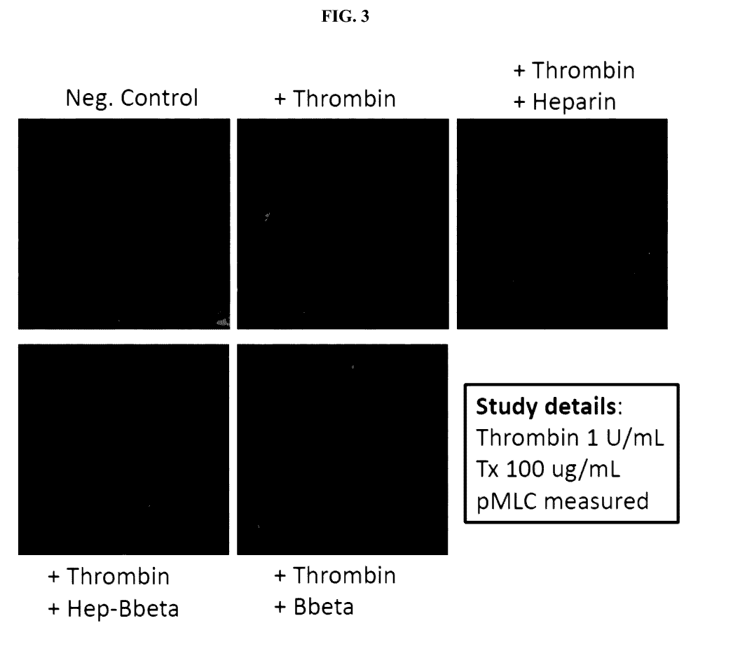

The plate was washed 4 times with 1×PBS. Each wash was for 10 minutes. The wells were then imaged using an EVOS fluorescence microscope. FIG. 3 shows that the bioconjugate of Example 1 preserves endothelial cell barrier function.

### Example 4. Fibrosis Model

The bioconjugates and compositions comprising the same as described herein can be tested for efficacy in fibrosis models known in the art see, e.g., Sadasivan, S. K., Fibrogenesis Tissue Repair, 2015, 8, 1.

Precision-cut liver slices of 150 μm thickness can be obtained from female C57BL/6 J mice. The slices can be cultured for 24 hours in media containing a cocktail of 10 nM each of TGF-β, PDGF, 5 μM each of lysophosphatidic acid and sphingosine 1 phosphate and 0.2 μg/ml of lipopolysaccharide along with 500 μM of palmitate and were analyzed for triglyceride accumulation, stress and inflammation, myofibroblast activation and extracellular matrix (ECM) accumulation. Incubation with the cocktail resulted in increased triglyceride accumulation, a hallmark of steatosis. The levels of Acta2, a hallmark of myofibroblast activation and the levels of inflammatory genes (IL-6, TNF-α and C-reactive protein) can be measured. In addition, this treatment may result in measurable levels of ECM markers—collagen, lumican and fibronectin.

This provides the experimental conditions required to induce fibrosis associated with steatohepatitis using physiologically relevant inducers. The system captures various aspects of the fibrosis process like steatosis, inflammation, stellate cell activation and ECM accumulation and serves as a platform to study the liver fibrosis in vitro and to screen bioconjugates for antifibrotic activity.

### Example 5. The Miles Assays—Vascular Leakage

Miles Assay A:

In the Miles Assay A, vascular barrier function is measured by extravasation of Evans blue dye from the vasculature into tissues. Evans blue binds to albumin, which cannot cross the endothelial barrier in a healthy animal. If the vascular barrier is compromised, then the blue dye will extravate from the vessels into tissues. Tissues can then be isolated, and the amount of blue dye in the tissue can be extracted and quantified by spectrophotometry.

Vascular leakage or endothelial barrier dysfunction can be initiated by a variety of agents, including lipopolysaccharide (LPS). Mice are IV injected with LPS. An agent designed to protect the endothelial barrier, such as a bioconjugate or a composition comprising the same as described herein, are then also IV injected. Next, Evans blue dye is injected into the animals. After approximately 1 hour, the animals are sacrificed and tissues including lung, brain, and intestines are harvested. The tissues are weighed, and the blue dye is extracted from the tissues with formic acid. The blue dye is then quantified by measuring its absorbance with a spectrophotometer and normalizing to the tissue weight.

It is contemplated that the bioconjugates or composition comprising the same will decrease vascular leak as determined by a reduced amount of blue dye that is found in tissues following initiation of vascular leak with a compound such as LPS. The assay may be further optimized by one of skill in the art.

Miles Assay B:

In the Miles Assay B, vascular barrier function is measured by extravasation of Evans blue dye from the vasculature into tissues. Evans blue binds to albumin, which cannot cross the endothelial barrier in a healthy animal. If the vascular barrier is compromised, then the blue dye will extravate from the vessels into tissues. Tissues can then be isolated, and the amount of blue dye in the tissue can be extracted and quantified by spectrophotometry.

Vascular leakage or endothelial barrier dysfunction can be initiated by a variety of agents, including vascular endothelial growth factor (VEGF). At time 0, rats were IV dosed with either PBS or test article. Immediately following the test article injective, animals received an IV injection of 2% Evans Blue dye. The dye injection was then followed by two intradermal injections of VEGF (200 ng) and PBS in a volume of 50 uL on each flank of the rat. At 15-20 minutes post Evans blue administration, the rats were euthanized and the dermal injection area was photographed. The skin covering the intradermal injection area was removed, inverted, and photographed. The intensity/extravasation of blue into the surrounding dermis in the VEGF injection site is compared to the PBS injection site and scored on a scale of 0 to 4. Additionally, skin plugs surrounding the intradermal injection site were taken and post Evans Blue administration, the rats will be euthanized. The dermal injection area will be photographed. The skin covering the ID injection area is then be removed, inverted and photographed. The intensity/extravasation of blue into the surrounding dermis in the VEGF injection site will be compared to the PBS injection site and scored according to the scale described below. Photographs will be taken of each animal. Skin plugs will also be taken and the blue dye was extracted with formamide and quantified by measuring absorbance with a spectrophotometer. It is contemplated that the assay may be further optimized by one of skill in the art. See, e.g., Palanki, et al. J. Med. Chem. 2007, 50, 4279-4294.

### Example 6. The Peritonitis Assay

Additionally, we have another assay that assesses peritonitis, another measure of vascular leak which specifically is measuring the ability of white blood cells to migrate into the peritoneal space.

Male C57BL/6 mice are dosed at time −2 to −5 minutes with PBS control or test article. At t=0 minutes, the animals then receive an intraperitoneal injection of thioglycolate to induce peritonitis. At 4 hours post thioglycolate induction, the animals are euthanized and a peritonineal lavage is performed. The neutrophil could in the peritoneal lavage is quantified by complete blood count analysis using a hematology analyzer. It is contemplated that the assay may be further optimized by one of skill in the art.

### Example 7. Renal Ischemia Reperfusion

This example shows that treatment with the bioconjugate as prepared in Example 1 immediately following renal reperfusion inhibits kidney damage. Kidney damage was assessed by measuring serum creatinine levels 24 hours post procedure, and by creatinine clearance measured at 24 hours and 7 days post procedure.

In this study, the ischemia time was reduced here to produce a more moderate injury, and the bioconjugate was delivered to the femoral vein, rather than directly to the renal artery, in order to reduce procedure time. See, Verma, et al. “Renal Endothelial Injury and Microvascular Dysfunction in Acute Kidney Injury.” Seminars in nephrology. Vol. 35. No. 1. WB Saunders, 2015 and Urbschat, et al. “Combined peri-ischemic administration of BP 15-42 in treating ischemia reperfusion injury of the mouse kidney.” Microvascular research 101 (2015): 48-54.

### Materials

1.1. Negative control: 1×PBS

1.2. Positive control: B-beta peptide (Urbschat 2015).

1.3. Test article: eHep-Bbeta

All test articles were formulated in 1×PBS at 5 mg/mL and 500 μL were delivered for an approximate dose of 10 mg/kg.

### Study Design Summary

1.4. Animals
1.4.1. Species: Rat
1.4.2. Strain: Sprague Dawley
1.4.3. Sex: Male
1.4.4. Total number of animals: 18
1.4.5. Animals per test article group: 6

1.5. Procedure
1.5.1. Prior to the procedure, blood was drawn in order to determine baseline serum creatinine levels.
1.5.2. Animals were anesthetized and the kidneys exposed.
1.5.3. One kidney from each animal was removed. The removed kidneys were saved in formalin for potentially histological analysis as healthy controls.
1.5.4. The remaining kidney was clamped at the renal pedical to obstruct blood flow to the kidney. The clamp remained in place for 30 minutes.
1.5.5. After 30 minutes, the clamp was removed, restoring blood flow to the kidney.
1.5.6. Immediately following clamp removal, test article was injected into the animal via the femoral vein.
1.5.7. Animals were closed and monitored during recovery for 24 hours.
1.5.8. 24 hours and 7 days post procedure, a blood sample was taken from each animal for serum creatinine measurement. Urine was also collected in order to assess creatinine clearance.
1.5.9. If creatinine clearance was positive in the positive control or test article at 7 days, the animal was survived until day 28, at which point creatinine clearance was again be measured. Animals were then euthanized and kidneys preserved for possible histological analysis.

### Analysis

1.6. Serum creatinine was measured in each animal at baseline (prior to procedure) and 24 hours post-procedure.
1.7. Creatinine clearance was measured at 1 and 7 days post procedure.
1.8. Serum creatinine levels in each animal at baseline and at 24 hours were compared using a paired t-test. This paired t-test determines if the serum creatinine levels changed in each animal as a result of the procedure.
1.9. Serum creatinine and creatinine clearance levels measured at 24 hours was compared between groups using an unpaired t-test. Data was considered statistically significant if the p-value was less than 0.05.

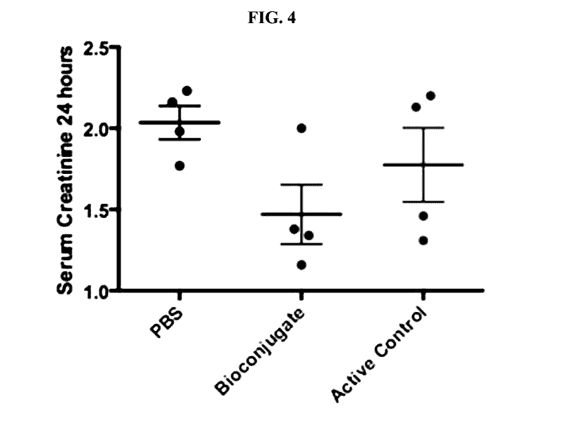

FIG. 4 shows that the bioconjugate as described in Example 1 protects from renal damage upon reperfusion better than active control (peptide alone) in an acute renal ischemic model.

1. A bioconjugate comprising a glycan and at least one peptide comprising a VE-Cadherin binding unit conjugated thereto.

2. The bioconjugate of claim 1, wherein the peptide comprises an amino acid sequence GHRPLDKKREEAPSLRPA (SEQ ID NO:2), or an amino acid sequence having one, two, or three amino acid addition, deletion and/or substitution(s) therefrom.

3. The bioconjugate of claim 1, wherein the peptide comprises up to about 50, or about 40, or about 30, or about 20 amino acids.

4. The bioconjugate of claim 1, wherein the peptide comprises an amino acid sequence GHRPLDKKREEAPSLRPAPPPISGGGYR (SEQ ID NO:3), or an amino acid sequence having one, two, or three amino acid addition, deletion and/or substitution therefrom.

5. The bioconjugate of claim 1, further comprising at least one selectin-binding unit, ICAM-binding unit, VCAM-binding unit, and/or collagen-binding unit.

6. The bioconjugate of claim 1, wherein the glycan is selected from the group consisting of alginate, chondroitin, chondroitin sulfate, dermatan, dermatan sulfate, heparan, heparan sulfate, heparin, dextran, dextran sulfate, and hyaluronan, or a derivative thereof.

7. The bioconjugate of claim 1, wherein the glycan is heparin.

8. The bioconjugate of claim 1, wherein the glycan is dermatan sulfate.

9. The bioconjugate of claim 1, wherein the glycan is hyaluronan.

10. The bioconjugate of claim 1, comprising from 1 to about 25 peptides, or from about 5 to about 25 peptides, or from about 1 to about 15 peptides, or about 2 peptides, or about 5 peptides, or about 10 peptides, or about 15 peptides.

11. The bioconjugate of claim 1, comprising from 50 to about 80 peptides, or from about 60 to about 70 peptides.

12. The bioconjugate of claim 1, wherein the glycan comprises:
a) from about 1 to about 75 percent (%) functionalization,
b) from about 5 to about 30 percent (%) functionalization,
c) from about 10 to about 40 percent (%) functionalization,
d) about 25 percent (%) functionalization, or
e) about 30 percent (%) functionalization,
wherein the percent (%) functionalization is determined by a percent of disaccharide units on the glycan which are functionalized with peptide.

13. The bioconjugate of claim 1, wherein the peptide is bound to the glycan via a spacer.

14. The bioconjugate of claim 13, wherein peptide is bound to the glycan via a spacer at the peptide N-terminus.

15. The bioconjugate of claim 13, wherein peptide is bound to the glycan via a spacer at the peptide C-terminus.

16. The bioconjugate of claim 1, wherein the peptide is bound to the glycan via a spacer and the spacer comprises between about 5 to about 50 carbon atoms.

17. The bioconjugate of claim 1, wherein the spacer comprises one or more amino acids selected from the group consisting of glycine, alanine, arginine, lysine and serine.

18. The bioconjugate of claim 17, wherein the spacer is selected from the group consisting of glycine, glycine-glycine, serine-glycine, lysine-arginine, arginine-arginine, and glycine-serine-glycine.

19. The bioconjugate of claim 1, wherein the peptide is bound to the glycan via a spacer and the spacer is branched.

20. A composition comprising the bioconjugate of claim 1 and one or more bioconjugates selected from the group consisting of:
a) a bioconjugate comprising a glycan and at least one peptide comprising a selectin-binding unit;
b) a bioconjugate comprising a glycan and at least one peptide comprising a ICAM-binding unit;
c) a bioconjugate comprising a glycan and at least one peptide comprising a VCAM-binding unit; and
d) a bioconjugate comprising a glycan and at least one peptide comprising a collagen-binding unit.

21. A composition comprising the bioconjugate of claim 1 and a bioconjugate comprising a glycan and at least one peptide comprising a collagen-binding unit.

22. A composition comprising the bioconjugate of claim 1, wherein the average number of peptide(s) per glycan is less than about 30.

23. A composition comprising the bioconjugate of claim 1, wherein the average percent functionalization of glycan with peptide(s) per about 30%.

24. A composition comprising the bioconjugate of claim 1, wherein the average number of peptide(s) per glycan is from about 5 to about 25.

25. A composition comprising the bioconjugate of claim 1, wherein the average number of peptide(s) per glycan is about 7.

26. A pharmaceutical composition comprising the bioconjugate of claim 1.

27. A method for maintaining endothelial integrity in a patient in need thereof, comprising administering to the patient an effective amount of the bioconjugate of claim 1.

28. A method for treating a patient suffering from a disease associated with endothelial dysfunction comprising administering to the patient an effective amount of the bioconjugate of claim 1.

29-40. (canceled)

41. A method for preventing or reducing inflammation at a vascular site in a patient, wherein the site (a) comprises permeated endothelial lining or damaged endothelial cells, and (b) is not undergoing or recovering from a vascular intervention procedure, the method comprising administering to the patient an effective amount of the bioconjugate of claim 1.

42. (canceled)

43. A method for treating or preventing ischemic reperfusion injury in a patient in need thereof, comprising administering to the patient an effective amount of the bioconjugate of claim 1.

44-46. (canceled)

47. A method of treating a fibrotic disease in a patient in need thereof, comprising administering to the patient an effective amount of the bioconjugate of claim 1.

48-51. (canceled)

52. A method for treating a disease or disorder selected from the group consisting of osteoarthritis, cancer, neointimal hyperplasia (peripheral & coronary), an ophthalmologic disease or disorder, tissue scarring, acute systemic disorders, chronic wounds, ischemia/reperfusion injury, central nervous system (CNS) diseases, fibrotic conditions, and vasculitis, the method comprising administering to the patient an effective amount of the bioconjugate of claim 1.


SEQUENCE LISTING (206 sequences)

SEQ ID NO: 1 - protein, 16 aa, Artificial Sequence
  Description of Artificial Sequence: Synthetic peptide
PSLRPAPPPISGGGYR

SEQ ID NO: 2 - protein, 18 aa, Artificial Sequence
  Description of Artificial Sequence: Synthetic peptide
  MOD_RES (1)..(18): May be L- or D-amino acid
GHRPLDKKREEAPSLRPA

SEQ ID NO: 3 - protein, 28 aa, Artificial Sequence
  Description of Artificial Sequence: Synthetic peptide
GHRPLDKKREEAPSLRPAPPPISGGGYR

SEQ ID NO: 4 - skipped

SEQ ID NO: 5 - protein, 17 aa, Artificial Sequence
  Description of Artificial Sequence: Synthetic peptide
APSLRPAPPPISGGGYR

SEQ ID NO: 6 - protein, 18 aa, Artificial Sequence
  Description of Artificial Sequence: Synthetic peptide
AAPSLRPAPPPISGGGYR

SEQ ID NO: 7 - protein, 19 aa, Artificial Sequence
  Description of Artificial Sequence: Synthetic peptide
RAAPSLRPAPPPISGGGYR

SEQ ID NO: 8 - protein, 19 aa, Artificial Sequence
  Description of Artificial Sequence: Synthetic peptide
PSLRPAPPPISGGGYRGSG

SEQ ID NO: 9 - protein, 20 aa, Artificial Sequence
  Description of Artificial Sequence: Synthetic peptide
APSLRPAPPPISGGGYRGSG

SEQ ID NO: 10 - protein, 21 aa, Artificial Sequence
  Description of Artificial Sequence: Synthetic peptide
AAPSLRPAPPPISGGGYRGSG

SEQ ID NO: 11 - protein, 22 aa, Artificial Sequence
  Description of Artificial Sequence: Synthetic peptide
RAAPSLRPAPPPISGGGYRGSG

SEQ ID NO: 12 - protein, 13 aa, Artificial Sequence
  Description of Artificial Sequence: Synthetic peptide
  MOD_RES (7)..(7): 6-aminohexanoic acid
CRVDAEXRVDAEC

SEQ ID NO: 13 - protein, 16 aa, Artificial Sequence
  Description of Artificial Sequence: Synthetic peptide
  MOD_RES (7)..(7): 6-aminohexanoic acid
CRVDAEXRVDAECGSG

SEQ ID NO: 14 - protein, 31 aa, Artificial Sequence
  Description of Artificial Sequence: Synthetic polypeptide
GHRPLDKKREEAPSLRPAPPPISGGGYRGSG

SEQ ID NO: 15 - protein, 27 aa, Artificial Sequence
  Description of Artificial Sequence: Synthetic peptide
GHRPLDKKREEAPSLRPAPPPISGGGY

SEQ ID NO: 16 - protein, 26 aa, Artificial Sequence
  Description of Artificial Sequence: Synthetic peptide
GHRPLDKKREEAPSLRPAPPPISGGG

SEQ ID NO: 17 - protein, 25 aa, Artificial Sequence
  Description of Artificial Sequence: Synthetic peptide
GHRPLDKKREEAPSLRPAPPPISGG

SEQ ID NO: 18 - protein, 24 aa, Artificial Sequence
  Description of Artificial Sequence: Synthetic peptide
GHRPLDKKREEAPSLRPAPPPISG

SEQ ID NO: 19 - protein, 23 aa, Artificial Sequence
  Description of Artificial Sequence: Synthetic peptide
GHRPLDKKREEAPSLRPAPPPIS

SEQ ID NO: 20 - protein, 22 aa, Artificial Sequence
  Description of Artificial Sequence: Synthetic peptide
GHRPLDKKREEAPSLRPAPPPI

SEQ ID NO: 21 - protein, 21 aa, Artificial Sequence
  Description of Artificial Sequence: Synthetic peptide
GHRPLDKKREEAPSLRPAPPP

SEQ ID NO: 22 - protein, 20 aa, Artificial Sequence
  Description of Artificial Sequence: Synthetic peptide
GHRPLDKKREEAPSLRPAPP

SEQ ID NO: 23 - protein, 19 aa, Artificial Sequence
  Description of Artificial Sequence: Synthetic peptide
GHRPLDKKREEAPSLRPAP

SEQ ID NO: 24 - protein, 17 aa, Artificial Sequence
  Description of Artificial Sequence: Synthetic peptide
GHRPLDKKREEAPSLRP

SEQ ID NO: 25 - protein, 16 aa, Artificial Sequence
  Description of Artificial Sequence: Synthetic peptide
GHRPLDKKREEAPSLR

SEQ ID NO: 26 - protein, 15 aa, Artificial Sequence
  Description of Artificial Sequence: Synthetic peptide
GHRPLDKKREEAPSL

SEQ ID NO: 27 - protein, 14 aa, Artificial Sequence
  Description of Artificial Sequence: Synthetic peptide
GHRPLDKKREEAPS

SEQ ID NO: 28 - protein, 13 aa, Artificial Sequence
  Description of Artificial Sequence: Synthetic peptide
GHRPLDKKREEAP

SEQ ID NO: 29 - protein, 12 aa, Artificial Sequence
  Description of Artificial Sequence: Synthetic peptide
GHRPLDKKREEA

SEQ ID NO: 30 - protein, 11 aa, Artificial Sequence
  Description of Artificial Sequence: Synthetic peptide
GHRPLDKKREE

SEQ ID NO: 31 - protein, 10 aa, Artificial Sequence
  Description of Artificial Sequence: Synthetic peptide
GHRPLDKKRE

SEQ ID NO: 32 - protein, 9 aa, Artificial Sequence
  Description of Artificial Sequence: Synthetic peptide
GHRPLDKKR

SEQ ID NO: 33 - protein, 21 aa, Artificial Sequence
  Description of Artificial Sequence: Synthetic peptide
GHRPLDKKREEAPSLRPAGSG

SEQ ID NO: 34 - protein, 18 aa, Artificial Sequence
  Description of Artificial Sequence: Synthetic peptide
  MOD_RES (1)..(1): Any natural or unnatural amino acid or absent
XHRPLDKKREEAPSLRPA

SEQ ID NO: 35 - protein, 18 aa, Artificial Sequence
  Description of Artificial Sequence: Synthetic peptide
  MOD_RES (2)..(2): Any natural or unnatural amino acid or absent
GXRPLDKKREEAPSLRPA

SEQ ID NO: 36 - protein, 18 aa, Artificial Sequence
  Description of Artificial Sequence: Synthetic peptide
  MOD_RES (3)..(3): Any natural or unnatural amino acid or absent
GHXPLDKKREEAPSLRPA

SEQ ID NO: 37 - protein, 18 aa, Artificial Sequence
  Description of Artificial Sequence: Synthetic peptide
  MOD_RES (4)..(4): Any natural or unnatural amino acid or absent
GHRXLDKKREEAPSLRPA

SEQ ID NO: 38 - protein, 18 aa, Artificial Sequence
  Description of Artificial Sequence: Synthetic peptide
  MOD_RES (5)..(5): Any natural or unnatural amino acid or absent
GHRPXDKKREEAPSLRPA

SEQ ID NO: 39 - protein, 18 aa, Artificial Sequence
  Description of Artificial Sequence: Synthetic peptide
  MOD_RES (6)..(6): Any natural or unnatural amino acid or absent
GHRPLXKKREEAPSLRPA

SEQ ID NO: 40 - protein, 18 aa, Artificial Sequence
  Description of Artificial Sequence: Synthetic peptide
  MOD_RES (7)..(7): Any natural or unnatural amino acid or absent
GHRPLDXKREEAPSLRPA

SEQ ID NO: 41 - protein, 18 aa, Artificial Sequence
  Description of Artificial Sequence: Synthetic peptide
  MOD_RES (8)..(8): Any natural or unnatural amino acid or absent
GHRPLDKXREEAPSLRPA

SEQ ID NO: 42 - protein, 18 aa, Artificial Sequence
  Description of Artificial Sequence: Synthetic peptide
  MOD_RES (9)..(9): Any natural or unnatural amino acid or absent
GHRPLDKKXEEAPSLRPA

SEQ ID NO: 43 - protein, 18 aa, Artificial Sequence
  Description of Artificial Sequence: Synthetic peptide
  MOD_RES (10)..(10): Any natural or unnatural amino acid or absent
GHRPLDKKRXEAPSLRPA

SEQ ID NO: 44 - protein, 18 aa, Artificial Sequence
  Description of Artificial Sequence: Synthetic peptide
  MOD_RES (11)..(11): Any natural or unnatural amino acid or absent
GHRPLDKKREXAPSLRPA

SEQ ID NO: 45 - protein, 18 aa, Artificial Sequence
  Description of Artificial Sequence: Synthetic peptide
  MOD_RES (12)..(12): Any natural or unnatural amino acid or absent
GHRPLDKKREEXASLRPA

SEQ ID NO: 46 - protein, 18 aa, Artificial Sequence
  Description of Artificial Sequence: Synthetic peptide
  MOD_RES (13)..(13): Any natural or unnatural amino acid or absent
GHRPLDKKREEAXSLRPA

SEQ ID NO: 47 - protein, 18 aa, Artificial Sequence
  Description of Artificial Sequence: Synthetic peptide
  MOD_RES (14)..(14): Any natural or unnatural amino acid or absent
GHRPLDKKREEAPXLRPA

SEQ ID NO: 48 - protein, 18 aa, Artificial Sequence
  Description of Artificial Sequence: Synthetic peptide
  MOD_RES (15)..(15): Any natural or unnatural amino acid or absent
GHRPLDKKREEAPSXRPA

SEQ ID NO: 49 - protein, 18 aa, Artificial Sequence
  Description of Artificial Sequence: Synthetic peptide
  MOD_RES (16)..(16): Any natural or unnatural amino acid or absent
GHRPLDKKREEAPSLXPA

SEQ ID NO: 50 - protein, 18 aa, Artificial Sequence
  Description of Artificial Sequence: Synthetic peptide
  MOD_RES (17)..(17): Any natural or unnatural amino acid or absent
GHRPLDKKREEAPSLRXA

SEQ ID NO: 51 - protein, 18 aa, Artificial Sequence
  Description of Artificial Sequence: Synthetic peptide
  MOD_RES (18)..(18): Any natural or unnatural amino acid or absent
GHRPLDKKREEAPSLRAX

SEQ ID NO: 52 - protein, 18 aa, Artificial Sequence
  Description of Artificial Sequence: Synthetic peptide
AHRPLDKKREEAPSLRPA

SEQ ID NO: 53 - protein, 18 aa, Artificial Sequence
  Description of Artificial Sequence: Synthetic peptide
GARPLDKKREEAPSLRPA

SEQ ID NO: 54 - protein, 18 aa, Artificial Sequence
  Description of Artificial Sequence: Synthetic peptide
GHAPLDKKREEAPSLRPA

SEQ ID NO: 55 - protein, 18 aa, Artificial Sequence
  Description of Artificial Sequence: Synthetic peptide
GHRALDKKREEAPSLRPA

SEQ ID NO: 56 - protein, 18 aa, Artificial Sequence
  Description of Artificial Sequence: Synthetic peptide
GHRPADKKREEAPSLRPA

SEQ ID NO: 57 - protein, 18 aa, Artificial Sequence
  Description of Artificial Sequence: Synthetic peptide
GHRPLAKKREEAPSLRPA

SEQ ID NO: 58 - protein, 18 aa, Artificial Sequence
  Description of Artificial Sequence: Synthetic peptide
GHRPLDAKREEAPSLRPA

SEQ ID NO: 59 - protein, 18 aa, Artificial Sequence
  Description of Artificial Sequence: Synthetic peptide
GHRPLDKAREEAPSLRPA

SEQ ID NO: 60 - protein, 18 aa, Artificial Sequence
  Description of Artificial Sequence: Synthetic peptide
GHRPLDKKAEEAPSLRPA

SEQ ID NO: 61 - protein, 18 aa, Artificial Sequence
  Description of Artificial Sequence: Synthetic peptide
GHRPLDKKRAEAPSLRPA

SEQ ID NO: 62 - protein, 18 aa, Artificial Sequence
  Description of Artificial Sequence: Synthetic peptide
GHRPLDKKREAAPSLRPA

SEQ ID NO: 63 - protein, 18 aa, Artificial Sequence
  Description of Artificial Sequence: Synthetic peptide
GHRPLDKKREEAASLRPA

SEQ ID NO: 64 - protein, 18 aa, Artificial Sequence
  Description of Artificial Sequence: Synthetic peptide
GHRPLDKKREEAPALRPA

SEQ ID NO: 65 - protein, 18 aa, Artificial Sequence
  Description of Artificial Sequence: Synthetic peptide
GHRPLDKKREEAPSARPA

SEQ ID NO: 66 - protein, 18 aa, Artificial Sequence
  Description of Artificial Sequence: Synthetic peptide
GHRPLDKKREEAPSLAPA

SEQ ID NO: 67 - protein, 18 aa, Artificial Sequence
  Description of Artificial Sequence: Synthetic peptide
GHRPLDKKREEAPSLRAA

SEQ ID NO: 68 - protein, 18 aa, Artificial Sequence
  Description of Artificial Sequence: Synthetic peptide
GHRPLNKKREEAPSLRPA

SEQ ID NO: 69 - protein, 18 aa, Artificial Sequence
  Description of Artificial Sequence: Synthetic peptide
GHRPLDKKRQEAPSLRPA

SEQ ID NO: 70 - protein, 18 aa, Artificial Sequence
  Description of Artificial Sequence: Synthetic peptide
GHRPLDKKREQAPSLRPA

SEQ ID NO: 71 - protein, 18 aa, Artificial Sequence
  Description of Artificial Sequence: Synthetic peptide
GHRPLDKKRQQAPSLRPA

SEQ ID NO: 72 - protein, 18 aa, Artificial Sequence
  Description of Artificial Sequence: Synthetic peptide
GHRPLNKKRQEAPSLRPA

SEQ ID NO: 73 - protein, 18 aa, Artificial Sequence
  Description of Artificial Sequence: Synthetic peptide
GHRPLNKKREQAPSLRPA

SEQ ID NO: 74 - protein, 18 aa, Artificial Sequence
  Description of Artificial Sequence: Synthetic peptide
GHRPLNKKRQQAPSLRPA

SEQ ID NO: 75 - protein, 7 aa, Artificial Sequence
  Description of Artificial Sequence: Synthetic peptide
TLTYTWS

SEQ ID NO: 76 - protein, 10 aa, Artificial Sequence
  Description of Artificial Sequence: Synthetic peptide
TLTYTWSGSG

SEQ ID NO: 77 - protein, 7 aa, Artificial Sequence
  Description of Artificial Sequence: Synthetic peptide
KLWVLPK

SEQ ID NO: 78 - protein, 18 aa, Artificial Sequence
  Description of Artificial Sequence: Synthetic peptide
RRANAALKAGELYKSILY

SEQ ID NO: 79 - protein, 9 aa, Artificial Sequence
  Description of Artificial Sequence: Synthetic peptide
GELYKSILY

SEQ ID NO: 80 - protein, 18 aa, Artificial Sequence
  Description of Artificial Sequence: Synthetic peptide
RRANAALKAGELYKCILY

SEQ ID NO: 81 - protein, 9 aa, Artificial Sequence
  Description of Artificial Sequence: Synthetic peptide
GELYKCILY

SEQ ID NO: 82 - protein, 9 aa, Artificial Sequence
  Description of Artificial Sequence: Synthetic peptide
RLDGNEIKR

SEQ ID NO: 83 - protein, 13 aa, Artificial Sequence
  Description of Artificial Sequence: Synthetic peptide
AHEEISTTNEGVM

SEQ ID NO: 84 - protein, 28 aa, Artificial Sequence
  Description of Artificial Sequence: Synthetic peptide
NGVFKYRPRYFLYKHAYFYPPLKRFPVQ

SEQ ID NO: 85 - protein, 10 aa, Artificial Sequence
  Description of Artificial Sequence: Synthetic peptide
CQDSETRTFY

SEQ ID NO: 86 - protein, 7 aa, Artificial Sequence
  Description of Artificial Sequence: Synthetic peptide
TKKTLRT

SEQ ID NO: 87 - protein, 28 aa, Artificial Sequence
  Description of Artificial Sequence: Synthetic peptide
GLRSKSKKFRRPDIQYPDATDEDITSHM

SEQ ID NO: 88 - protein, 7 aa, Artificial Sequence
  Description of Artificial Sequence: Synthetic peptide
SQNPVQP

SEQ ID NO: 89 - protein, 11 aa, Artificial Sequence
  Description of Artificial Sequence: Synthetic peptide
SYIRIADTNIT

SEQ ID NO: 90 - protein, 8 aa, Artificial Sequence
  Description of Artificial Sequence: Synthetic peptide
KELNLVYT

SEQ ID NO: 91 - protein, 4 aa, Artificial Sequence
  Description of Artificial Sequence: Synthetic peptide
GSIT

SEQ ID NO: 92 - protein, 12 aa, Artificial Sequence
  Description of Artificial Sequence: Synthetic peptide
GSITTIDVPWNV

SEQ ID NO: 93 - protein, 9 aa, Artificial Sequence
  Description of Artificial Sequence: Synthetic peptide
GQLYKSILY

SEQ ID NO: 94 - protein, 18 aa, Artificial Sequence
  Description of Artificial Sequence: Synthetic peptide
RRANAALKAGQLYKSILY

SEQ ID NO: 95 - protein, 10 aa, Artificial Sequence
  Description of Artificial Sequence: Synthetic peptide
WREPSFCALS

SEQ ID NO: 96 - protein, 16 aa, Artificial Sequence
  Description of Artificial Sequence: Synthetic peptide
  MOD_RES (1)..(1): Beta-Ala
  MOD_RES (16)..(16): Biphenylalanine
AWHCTTKFPHHYCLYX

SEQ ID NO: 97 - protein, 17 aa, Artificial Sequence
  Description of Artificial Sequence: Synthetic peptide
  MOD_RES (1)..(1): Beta-Ala
  MOD_RES (17)..(17): Biphenylalanine
AHKCPWHLYTTHYCFTX

SEQ ID NO: 98 - protein, 15 aa, Artificial Sequence
  Description of Artificial Sequence: Synthetic peptide
  MOD_RES (1)..(1): Beta-Ala
AHKCPWHLYTHYCFT

SEQ ID NO: 99 - protein, 6 aa, Artificial Sequence
  Description of Artificial Sequence: Synthetic peptide
  MOD_RES (3)..(3): 4-Hydroxyproline
GRPGER

SEQ ID NO: 100 - protein, 6 aa, Artificial Sequence
  Description of Artificial Sequence: Synthetic peptide
  MOD_RES (3)..(3): 4-Hydroxyproline
GMPGER

SEQ ID NO: 101 - protein, 6 aa, Artificial Sequence
  Description of Artificial Sequence: Synthetic peptide
  MOD_RES (3)..(3): 4-Hydroxyproline
GLPGEN

SEQ ID NO: 102 - protein, 6 aa, Artificial Sequence
  Description of Artificial Sequence: Synthetic peptide
  MOD_RES (3)..(3): 4-Hydroxyproline
GLPGER

SEQ ID NO: 103 - protein, 6 aa, Artificial Sequence
  Description of Artificial Sequence: Synthetic peptide
GLKGEN

SEQ ID NO: 104 - protein, 15 aa, Artificial Sequence
  Description of Artificial Sequence: Synthetic peptide
  MOD_RES (3)..(3): 4-Hydroxyproline
  MOD_RES (12)..(12): 4-Hydroxyproline
GFPGERGVEGPPGPA

SEQ ID NO: 105 - protein, 11 aa, Artificial Sequence
  Description of Artificial Sequence: Synthetic peptide
HVWMQAPGGGK

SEQ ID NO: 106 - protein, 6 aa, Artificial Sequence
  Description of Artificial Sequence: Synthetic peptide
WYRGRL

SEQ ID NO: 107 - protein, 12 aa, Artificial Sequence
  Description of Artificial Sequence: Synthetic peptide
WTCSGDEYTWHC

SEQ ID NO: 108 - protein, 12 aa, Artificial Sequence
  Description of Artificial Sequence: Synthetic peptide
WTCVGDHKTWKC

SEQ ID NO: 109 - protein, 16 aa, Artificial Sequence
  Description of Artificial Sequence: Synthetic peptide
QWHCTTRFPHHYCLYG

SEQ ID NO: 110 - protein, 17 aa, Artificial Sequence
  Description of Artificial Sequence: Synthetic peptide
STWTWNGSAWTWNEGGK

SEQ ID NO: 111 - protein, 17 aa, Artificial Sequence
  Description of Artificial Sequence: Synthetic peptide
STWTWNGTNWTRNDGGK

SEQ ID NO: 112 - protein, 8 aa, Artificial Sequence
  Description of Artificial Sequence: Synthetic peptide
CVWLWEQC

SEQ ID NO: 113 - protein, 8 aa, Artificial Sequence
  Description of Artificial Sequence: Synthetic peptide
CVWLWENC

SEQ ID NO: 114 - protein, 8 aa, Artificial Sequence
  Description of Artificial Sequence: Synthetic peptide
CMTSPWRC

SEQ ID NO: 115 - protein, 18 aa, Artificial Sequence
  Description of Artificial Sequence: Synthetic peptide
CPGRVMHGLHLGDDEGPC

SEQ ID NO: 116 - protein, 7 aa, Artificial Sequence
  Description of Artificial Sequence: Synthetic peptide
KLWLLPK

SEQ ID NO: 117 - protein, 9 aa, Artificial Sequence
  Description of Artificial Sequence: Synthetic peptide
LSELRLHEN

SEQ ID NO: 118 - protein, 9 aa, Artificial Sequence
  Description of Artificial Sequence: Synthetic peptide
LTELHLDNN

SEQ ID NO: 119 - protein, 9 aa, Artificial Sequence
  Description of Artificial Sequence: Synthetic peptide
LSELRLHNN

SEQ ID NO: 120 - protein, 9 aa, Artificial Sequence
  Description of Artificial Sequence: Synthetic peptide
LSELRLHAN

SEQ ID NO: 121 - protein, 9 aa, Artificial Sequence
  Description of Artificial Sequence: Synthetic peptide
LRELHLNNN

SEQ ID NO: 122 - protein, 12 aa, Artificial Sequence
  Description of Artificial Sequence: Synthetic peptide
RVMHGLHLGDDE

SEQ ID NO: 123 - protein, 12 aa, Artificial Sequence
  Description of Artificial Sequence: Synthetic peptide
EDDGLHLGHMVR

SEQ ID NO: 124 - protein, 12 aa, Artificial Sequence
  Description of Artificial Sequence: Synthetic peptide
RVMHGLHLGNNQ

SEQ ID NO: 125 - protein, 12 aa, Artificial Sequence
  Description of Artificial Sequence: Synthetic peptide
QNNGLHLGHMVR

SEQ ID NO: 126 - protein, 12 aa, Artificial Sequence
  Description of Artificial Sequence: Synthetic peptide
GQLYKSILYGSG

SEQ ID NO: 127 - protein, 11 aa, Artificial Sequence
  Description of Artificial Sequence: Synthetic peptide
GSGQLYKSILY

SEQ ID NO: 128 - protein, 12 aa, Artificial Sequence
  Description of Artificial Sequence: Synthetic peptide
GSGGQLYKSILY

SEQ ID NO: 129 - protein, 8 aa, Artificial Sequence
  Description of Artificial Sequence: Synthetic peptide
KQLNLVYT

SEQ ID NO: 130 - protein, 8 aa, Artificial Sequence
  Description of Artificial Sequence: Synthetic peptide
CVWLWQQC

SEQ ID NO: 131 - protein, 10 aa, Artificial Sequence
  Description of Artificial Sequence: Synthetic peptide
WREPSFSALS

SEQ ID NO: 132 - protein, 28 aa, Artificial Sequence
  Description of Artificial Sequence: Synthetic peptide
GHRPLNKKRQQAPSLRPAPPPISGGGYR

SEQ ID NO: 133 - protein, 7 aa, Artificial Sequence
  Description of Artificial Sequence: Synthetic peptide
IELLQAR

SEQ ID NO: 134 - protein, 10 aa, Artificial Sequence
  Description of Artificial Sequence: Synthetic peptide
IELLQARGSC

SEQ ID NO: 135 - protein, 7 aa, Artificial Sequence
  Description of Artificial Sequence: Synthetic peptide
IDLMQAR

SEQ ID NO: 136 - protein, 10 aa, Artificial Sequence
  Description of Artificial Sequence: Synthetic peptide
IDLMQARGSC

SEQ ID NO: 137 - protein, 12 aa, Artificial Sequence
  Description of Artificial Sequence: Synthetic peptide
QITWAQLWNMMK

SEQ ID NO: 138 - protein, 15 aa, Artificial Sequence
  Description of Artificial Sequence: Synthetic peptide
QITWAQLWNMMKGSC

SEQ ID NO: 139 - protein, 21 aa, Artificial Sequence
  Description of Artificial Sequence: Synthetic peptide
LRRASLGDGDITWDQLWDLMK

SEQ ID NO: 140 - protein, 12 aa, Artificial Sequence
  Description of Artificial Sequence: Synthetic peptide
HITWDQLWNVMN

SEQ ID NO: 141 - protein, 20 aa, Artificial Sequence
  Description of Artificial Sequence: Synthetic peptide
YGNSNITWDQLWSIMNRQTT

SEQ ID NO: 142 - protein, 20 aa, Artificial Sequence
  Description of Artificial Sequence: Synthetic peptide
WTDTHITWDQLWHFMNMGEQ

SEQ ID NO: 143 - protein, 20 aa, Artificial Sequence
  Description of Artificial Sequence: Synthetic peptide
EPWDQITWDQLWIIMNNGDG

SEQ ID NO: 144 - protein, 12 aa, Artificial Sequence
  Description of Artificial Sequence: Synthetic peptide
HITWDQLWLMMS

SEQ ID NO: 145 - protein, 12 aa, Artificial Sequence
  Description of Artificial Sequence: Synthetic peptide
DLTWEGLWILMT

SEQ ID NO: 146 - protein, 12 aa, Artificial Sequence
  Description of Artificial Sequence: Synthetic peptide
RGVWGGLWSMTW

SEQ ID NO: 147 - protein, 12 aa, Artificial Sequence
  Description of Artificial Sequence: Synthetic peptide
DYSWHDLWFMMS

SEQ ID NO: 148 - protein, 18 aa, Artificial Sequence
  Description of Artificial Sequence: Synthetic peptide
KKEDWLALWRIMSVPDEN

SEQ ID NO: 149 - protein, 13 aa, Artificial Sequence
  Description of Artificial Sequence: Synthetic peptide
RNMSWLELWEHMK

SEQ ID NO: 150 - protein, 13 aa, Artificial Sequence
  Description of Artificial Sequence: Synthetic peptide
KEQQWRNLWKMMS

SEQ ID NO: 151 - protein, 18 aa, Artificial Sequence
  Description of Artificial Sequence: Synthetic peptide
SQVTWNDLWSVMNPEVVN

SEQ ID NO: 152 - protein, 13 aa, Artificial Sequence
  Description of Artificial Sequence: Synthetic peptide
RSLSWLQLWDWMK

SEQ ID NO: 153 - protein, 12 aa, Artificial Sequence
  Description of Artificial Sequence: Synthetic peptide
DITWDQLWDLMK

SEQ ID NO: 154 - protein, 12 aa, Artificial Sequence
  Description of Artificial Sequence: Synthetic peptide
DITWDELWKIMN

SEQ ID NO: 155 - protein, 12 aa, Artificial Sequence
  Description of Artificial Sequence: Synthetic peptide
DYTWFELWDMMQ

SEQ ID NO: 156 - protein, 12 aa, Artificial Sequence
  Description of Artificial Sequence: Synthetic peptide
DMTHDLWLTLMS

SEQ ID NO: 157 - protein, 12 aa, Artificial Sequence
  Description of Artificial Sequence: Synthetic peptide
EITWDQLWEVMN

SEQ ID NO: 158 - protein, 12 aa, Artificial Sequence
  Description of Artificial Sequence: Synthetic peptide
HVSWEQLWDIMN

SEQ ID NO: 159 - protein, 12 aa, Artificial Sequence
  Description of Artificial Sequence: Synthetic peptide
HITWDQLWRIMT

SEQ ID NO: 160 - protein, 12 aa, Artificial Sequence
  Description of Artificial Sequence: Synthetic peptide
DISWDDLWIMMN

SEQ ID NO: 161 - protein, 12 aa, Artificial Sequence
  Description of Artificial Sequence: Synthetic peptide
QITWDQLWDLMY

SEQ ID NO: 162 - protein, 18 aa, Artificial Sequence
  Description of Artificial Sequence: Synthetic peptide
AEWTWDQLWHVMNPAESQ

SEQ ID NO: 163 - protein, 14 aa, Artificial Sequence
  Description of Artificial Sequence: Synthetic peptide
HRAEWLALWEQMSP

SEQ ID NO: 164 - protein, 14 aa, Artificial Sequence
  Description of Artificial Sequence: Synthetic peptide
KKEDWLALWRIMSV

SEQ ID NO: 165 - protein, 13 aa, Artificial Sequence
  Description of Artificial Sequence: Synthetic peptide
KRKQWIELWNIMS

SEQ ID NO: 166 - protein, 12 aa, Artificial Sequence
  Description of Artificial Sequence: Synthetic peptide
WKLDTLDMIWQD

SEQ ID NO: 167 - protein, 19 aa, Artificial Sequence
  Description of Artificial Sequence: Synthetic peptide
HITWDQLWNVMLRRAASLG

SEQ ID NO: 168 - protein, 14 aa, Artificial Sequence
  Description of Artificial Sequence: Synthetic peptide
NAFKILVVITFGEK

SEQ ID NO: 169 - protein, 17 aa, Artificial Sequence
  Description of Artificial Sequence: Synthetic peptide
NAFKILVVITFGEKGSC

SEQ ID NO: 170 - protein, 6 aa, Artificial Sequence
  Description of Artificial Sequence: Synthetic peptide
ITDGEA

SEQ ID NO: 171 - protein, 9 aa, Artificial Sequence
  Description of Artificial Sequence: Synthetic peptide
ITDGEAGSC

SEQ ID NO: 172 - protein, 6 aa, Artificial Sequence
  Description of Artificial Sequence: Synthetic peptide
DGEATD

SEQ ID NO: 173 - protein, 9 aa, Artificial Sequence
  Description of Artificial Sequence: Synthetic peptide
DGEATDGSC

SEQ ID NO: 174 - protein, 14 aa, Artificial Sequence
  Description of Artificial Sequence: Synthetic peptide
EWCEYLGGYLRYCA

SEQ ID NO: 175 - protein, 16 aa, Artificial Sequence
  Description of Artificial Sequence: Synthetic peptide
FEGFSFLAFEDFVSSI

SEQ ID NO: 176 - protein, 17 aa, Artificial Sequence
  Description of Artificial Sequence: Synthetic peptide
NNQKIVNLKEKVAQLEA

SEQ ID NO: 177 - protein, 17 aa, Artificial Sequence
  Description of Artificial Sequence: Synthetic peptide
NNQKIVNIKEKVAQIEA

SEQ ID NO: 178 - protein, 17 aa, Artificial Sequence
  Description of Artificial Sequence: Synthetic peptide
NNQKLVNIKEKVAQIEA

SEQ ID NO: 179 - protein, 7 aa, Artificial Sequence
  Description of Artificial Sequence: Synthetic peptide
YPASYQR

SEQ ID NO: 180 - protein, 7 aa, Artificial Sequence
  Description of Artificial Sequence: Synthetic peptide
YQATPLP

SEQ ID NO: 181 - protein, 7 aa, Artificial Sequence
  Description of Artificial Sequence: Synthetic peptide
GSLLSAA

SEQ ID NO: 182 - protein, 7 aa, Artificial Sequence
  Description of Artificial Sequence: Synthetic peptide
FSPHSRT

SEQ ID NO: 183 - protein, 7 aa, Artificial Sequence
  Description of Artificial Sequence: Synthetic peptide
YPFLPTA

SEQ ID NO: 184 - protein, 7 aa, Artificial Sequence
  Description of Artificial Sequence: Synthetic peptide
GCKLCAQ

SEQ ID NO: 185 - DNA, 45 bp, Artificial Sequence
  Description of Artificial Sequence: Synthetic oligonucleotide
ggtcggggtgagtttcgtggtagggataattctgtttgggtggtt

SEQ ID NO: 186 - protein, 14 aa, Artificial Sequence
  Description of Artificial Sequence: Synthetic peptide
EWCEYLGGYLRCYA

SEQ ID NO: 187 - protein, 15 aa, Artificial Sequence
  Description of Artificial Sequence: Synthetic peptide
GRGEFRGRDNSVSVV

SEQ ID NO: 188 - protein, 10 aa, Artificial Sequence
  Description of Artificial Sequence: Synthetic peptide
QTSVSPSKVI

SEQ ID NO: 189 - protein, 10 aa, Artificial Sequence
  Description of Artificial Sequence: Synthetic peptide
PSKVILPRGG

SEQ ID NO: 190 - protein, 11 aa, Artificial Sequence
  Description of Artificial Sequence: Synthetic peptide
LPRGGSVLVTG

SEQ ID NO: 191 - protein, 21 aa, Artificial Sequence
  Description of Artificial Sequence: Synthetic peptide
QTSVSPSKVILPRGGSVLVTG

SEQ ID NO: 192 - protein, 10 aa, Artificial Sequence
  Description of Artificial Sequence: Synthetic peptide
YRLAIRLNER

SEQ ID NO: 193 - protein, 20 aa, Artificial Sequence
  Description of Artificial Sequence: Synthetic peptide
YRLAIRLNERRENLRIALRY

SEQ ID NO: 194 - protein, 10 aa, Artificial Sequence
  Description of Artificial Sequence: Synthetic peptide
RENLRIALRY

SEQ ID NO: 195 - protein, 16 aa, Artificial Sequence
  Description of Artificial Sequence: Synthetic peptide
RYGGGSIPPPAPRLSP

SEQ ID NO: 196 - protein, 18 aa, Artificial Sequence
  Description of Artificial Sequence: Synthetic peptide
APRLSPAEERKKDLPRHG

SEQ ID NO: 197 - protein, 28 aa, Artificial Sequence
  Description of Artificial Sequence: Synthetic peptide
RYGGGSIPPPAPRLSPAEERKKDLPRHG

SEQ ID NO: 198 - protein, 4 aa, Artificial Sequence
  Description of Artificial Sequence: Synthetic peptide
KGSG

SEQ ID NO: 199 - protein, 5 aa, Artificial Sequence
  Description of Artificial Sequence: Synthetic peptide
KKGSG

SEQ ID NO: 200 - protein, 4 aa, Artificial Sequence
  Description of Artificial Sequence: Synthetic peptide
KGSG

SEQ ID NO: 201 - protein, 6 aa, Artificial Sequence
  Description of Artificial Sequence: Synthetic peptide
KKKGSG

SEQ ID NO: 202 - protein, 4 aa, Artificial Sequence
  Description of Artificial Sequence: Synthetic peptide
GGGC

SEQ ID NO: 203 - protein, 4 aa, Artificial Sequence
  Description of Artificial Sequence: Synthetic peptide
GSGC

SEQ ID NO: 204 - protein, 4 aa, Artificial Sequence
  Description of Artificial Sequence: Synthetic peptide
GGGG

SEQ ID NO: 205 - protein, 5 aa, Artificial Sequence
  Description of Artificial Sequence: Synthetic peptide
GGGGG

SEQ ID NO: 206 - protein, 5 aa, Artificial Sequence
  Description of Artificial Sequence: Synthetic peptide
GSGSG